SEQUENCE LISTING (1 sequence)

SEQ ID NO: 1 - DNA, 49 bp, Artificial Sequence
  Oligonucleotide
aataaaatatctttattttcattacatctgtgtgttggttttttgtgtg

(19) United States
(12) Patent Application Publication
(10) Pub. No.: US 2023/0364209 A1
(43) Pub. Date: Nov. 16, 2023

(54) IMMUNOGENIC SCHISTOSOMA COMPOSITIONS

(71) Applicant: THE ROYAL INSTITUTION FOR THE ADVANCEMENT OF LEARNING/MCGILL UNIVERSITY, Montreal (CA)

(72) Inventors: Momar NDAO, Candiac (CA); Dilhan Jonathan PERERA, Montréal (CA)

(21) Appl. No.: 18/248,130

(22) PCT Filed: Oct. 8, 2021
(86) PCT No.: PCT/CA2021/051418

§ 371 Date: Apr. 6, 2023

Related U.S. Application Data
(60) Provisional application No. 63/089,710, filed on Oct. 9, 2020.

(51) Int. Cl. A61K 39/00; A61K 47/02; A61P 37/04

(52) U.S. Cl. CPC A61K 39/0003; A61K 47/02; A61P 37/04; A61K 2039/55566; A61K 2039/575

(57) ABSTRACT

The present disclosure provides an immunogenic composition comprising an emulsion of an epitope or a nucleic acid molecule. The emulsion comprises an oil phase and a water phase. The emulsion is an oil-in-water emulsion and/or a nanoemulsion. The epitope is present on a peptide or a polypeptide derived from Schistosoma sp. and can optionally be glycosylated. The immunogenic composition (which can be provided as a pharmaceutical composition or as a vaccine) can be used to prevent, treat or alleviation the symptoms of a Schistosoma sp. infection.

### CROSS-REFERENCE TO RELATED APPLICATION AND DOCUMENT

The present application claims priority from U.S. provisional application 63/089,710 filed on Oct. 9, 2020 and herewith incorporated in its entirety.

### TECHNOLOGICAL FIELD

The present disclosure concerns compositions comprising at least one Schistosoma protein epitope capable of eliciting an immune response in a host for use in the prevention or the treatment of a Schistosoma infection.

### BACKGROUND

Schistosomiasis (Bilharzia) is an underestimated parasitic disease for which over 800 million people are at risk. Adult worms themselves live around the host intestines causing little to no pathology. However, female worms lay up to 300 eggs per day, some of which will exit with the feces or urine depending on the species of Schistosoma, and others will become trapped in host tissues causing chronic pathology.

Praziquantel (PZQ) used for the treatment of schistosomiasis has a reported efficacy of 85-90%. Although it does not protect individuals from reinfection, or remove pre-existing egg deposition. To rectify these shortcomings and aid the interruption of schistosomiasis a vaccine is pertinent. In the 1990s, independent testing of six candidate Schistosoma mansoni (S. mansoni) antigens underwent protective studies organized by a UNDP/World Bank/WHO Special Programme for Research and Training in Tropical Diseases (TDR/WHO) committee. Although these trials resulted in protection, the WHO goal of 40% or greater protection was not met, headlining the need for possible adjuvanted formulations.

S. mansoni cathepsin B (SmCB) is the most abundant cysteine protease found in schistosomula and adult worm gut and somatic extracts. This protein is used for host blood molecule degradation and nutrient acquisition. RNA interference studies demonstrated that when cathepsin B transcript levels are suppressed resulting worms show significant growth retardation compared to control parasites. By targeting cathepsin B, reduced egg fitness has been demonstrated by our group, and parasite anti-fecundity has also been seen in other flukes.

It was previously shown that the immunogenic gut peptidase S. mansoni Cathepsin B (SmCB) can be used as a vaccine target, since it reduced worm parasite burden by 59% and 60% when adjuvanted with CpG dinucleotides, and Montanide ISA 720 VG, respectively (Ricciardi et al., 2015; Ricciardi et al., 2016).

It would be highly desirable to be provided with a more effective immunogenic composition against Schistosoma sp. (such as S. mansoni). Such immunogenic composition could provide an increased cell-mediate immune response against the pathogen.

### BRIEF SUMMARY

The present disclosure concern immunogenic compositions comprising a peptide or a polypeptide derived from Schistosoma sp. for eliciting an immune reaction (e.g., cellular and/or humoral) against Schistosoma sp.

According to a first aspect, the present disclosure provides an immunogenic composition comprising an emulsion of an epitope. The emulsion comprises an oil phase and a water phase. The emulsion is an oil-in-water emulsion and/or a nanoemulsion. The epitope is present on a peptide or a polypeptide derived from Schistosoma sp. and can optionally be glycosylated. In an embodiment, the oil phase comprises a squalene or a squalene derivative. In still another embodiment, the immunogenic composition further comprises an emulsifier. In still another embodiment, the emulsifier is a fatty acid ester. In a further embodiment, the fatty acid ester is a sorbitan fatty acid ester. In yet a further embodiment, the sorbitan fatty acid ester is sorbitan triolate. In another embodiment, the immunogenic composition further comprises a surfactant. In still a further embodiment, the surfactant is a non-ionic surfactant. In an embodiment, the non-ionic surfactant is a polysorbate. In still a further embodiment, the polysorbate is polysorbate 80. In an embodiment, the immunogenic composition further comprises a buffer. In still another embodiment, the buffer is a citrate buffer. In yet another embodiment, the citrate buffer is sodium citrate. In an embodiment, the peptide or the polypeptide is derived from Schistosoma mansoni. In yet another embodiment, the peptide or the polypeptide is cathepsin B, an immunogenic cathepsin B variant or an immunogenic cathepsin B fragment. In still a further embodiment, the peptide or the polypeptide is glycosylated.

According to a second aspect, the present disclosure provides an immunogenic composition comprising a nucleic acid molecule encoding cathepsin B, an immunogenic cathepsin B variant or an immunogenic cathepsin B fragment. In some embodiments, the nucleic acid molecule is an adenovirus-derived vector. In another embodiment, the immunogenic composition can comprise one or more viral particles. In yet additional embodiments, the immunogenic composition can comprise a polypeptide, wherein the polypeptide comprises cathepsin B, an immunogenic cathepsin B variant or an immunogenic cathepsin B fragment (which may, in some further embodiments, be glycosylated).

According to a third aspect, the present disclosure provides a pharmaceutical composition comprising the immunogenic composition described and an excipient. In an embodiment, the pharmaceutical composition is formulated for intramuscular administration.

According to a fourth aspect, the present disclosure provides a vaccine comprising the immunogenic composition described herein. In an embodiment, the vaccine is formulated for intramuscular administration.

According to a fifth aspect, the present disclosure provides a process for making an immunogenic composition. The process comprises admixing an emulsion as defined herein with an epitope as defined herein to make the immunogenic composition.

According to a sixth aspect, the present disclosure provides a process for making a vaccine, the process comprising admixing an emulsion as defined herein with an epitope as defined herein to make the vaccine.

According to a seventh aspect, the present disclosure provides a method of preventing a Schistosoma sp. infection in a subject. The method comprising administering at least one dose of the pharmaceutical composition or the vaccine described herein to the subject so as to prevent the Schistosoma sp. infection. In an embodiment, the method comprises administering a first priming dose and a second booster dose. In another embodiment, the method comprises administering a third booster dose. In still another embodiment, the method is for increasing a humoral response against Schistosoma sp. in the subject. In yet another embodiment, the method is for increasing the IgG titers response against Schistosoma sp. in the subject. In yet another embodiment, the method is for increasing a cell-mediated immune response against Schistosoma sp. in the subject. In yet a further embodiment, the method is for reducing the number of adult Schistosoma sp. worms in the subject, for reducing the number of Schistosoma sp. eggs in the subject, for reducing liver cirrhosis in the subject, for reducing liver granuloma formation in the subject, for reducing the number of hatched Schistosoma sp. parasites in the stool of the subject and/or for reducing IgGE sensitivity in the subject. In an embodiment, the subject is a mammal, such as, for example, a human. In still another embodiment, the Schistosoma sp. is Schistosoma mansoni.

According to an eight aspect, the present disclosure provides a method of treating a Schistosoma sp. infection or reducing a symptom associated with the Schistosoma sp. infection in a subject in need thereof. The method comprising administering at least one dose of the pharmaceutical composition or the vaccine described herein to the subject so as to prevent the Schistosoma sp. infection. In an embodiment, the method comprises administering a first priming dose and a second booster dose. In another embodiment, the method comprises administering a third booster dose. In still another embodiment, the method is for increasing a humoral response against Schistosoma sp. in the subject. In yet another embodiment, the method is for increasing the IgG titers response against Schistosoma sp. in the subject. In yet another embodiment, the method is for increasing a cell-mediated immune response against Schistosoma sp. in the subject. In yet a further embodiment, the method is for reducing the number of adult Schistosoma sp. worms in the subject, for reducing the number of Schistosoma sp. eggs in the subject, for reducing liver cirrhosis in the subject, for reducing liver granuloma formation in the subject, for reducing the number of hatched Schistosoma sp. parasites in the stool of the subject and/or for reducing IgGE sensitivity in the subject. In an embodiment, the subject is a mammal, such as, for example, a human. In still another embodiment, the Schistosoma sp. is Schistosoma mansoni.

According to a ninth aspect, the present disclosure provides the use of at least one dose of pharmaceutical composition or vaccine as described herein for preventing a Schistosoma sp. infection in a subject or for the manufacture of a medicament for preventing a Schistosoma sp. infection in a subject. The present disclosure also provide a pharmaceutical composition or a vaccine as described herein for preventing a Schistosoma sp. infection in a subject or for the manufacture of a medicament for preventing a Schistosoma sp. infection in a subject. In an embodiment, the pharmaceutical composition or the vaccine can be formulated a first priming dose and a second booster dose. In another embodiment, the pharmaceutical composition or the vaccine can be formulated as a third booster dose. In still another embodiment, the pharmaceutical composition or the vaccine can be used for increasing a humoral response against Schistosoma sp. in the subject. In yet another embodiment, the pharmaceutical composition or the vaccine can be used for increasing the IgG titers response against Schistosoma sp. in the subject. In yet another embodiment, the pharmaceutical composition or the vaccine can be used for increasing a cell-mediated immune response against Schistosoma sp. in the subject. In yet a further embodiment, the pharmaceutical composition or the vaccine can be used for reducing the number of adult Schistosoma sp. worms in the subject, for reducing the number of Schistosoma sp. eggs in the subject, for reducing liver cirrhosis in the subject, for reducing liver granuloma formation in the subject, for reducing the number of hatched Schistosoma sp. parasites in the stool of the subject and/or for reducing IgGE sensitivity in the subject. In an embodiment, the subject is a mammal, such as, for example, a human. In still another embodiment, the Schistosoma sp. is Schistosoma mansoni.

According to a tenth aspect, the present disclosure provides the use of at least one dose of pharmaceutical composition or vaccine as described herein for treating a Schistosoma sp. infection or reducing a symptom associated with the Schistosoma sp. infection in a subject or for the manufacture of a medicament for treating a Schistosoma sp. infection or reducing a symptom associated with the Schistosoma sp. infection in a subject. The present disclosure also provide a pharmaceutical composition or a vaccine as described herein treating a Schistosoma sp. infection or reducing a symptom associated with the Schistosoma sp. infection in a subject or for the manufacture of a treating a Schistosoma sp. infection or reducing a symptom associated with the Schistosoma sp. infection in a subject. In an embodiment, the pharmaceutical composition or the vaccine can be formulated a first priming dose and a second booster dose. In another embodiment, the pharmaceutical composition or the vaccine can be formulated as a third booster dose. In still another embodiment, the pharmaceutical composition or the vaccine can be used for increasing a humoral response against Schistosoma sp. in the subject. In yet another embodiment, the pharmaceutical composition or the vaccine can be used for increasing the IgG titers response against Schistosoma sp. in the subject. In yet another embodiment, the pharmaceutical composition or the vaccine can be used for increasing a cell-mediated immune response against Schistosoma sp. in the subject. In yet a further embodiment, the pharmaceutical composition or the vaccine can be used for reducing the number of adult Schistosoma sp. worms in the subject, for reducing the number of Schistosoma sp. eggs in the subject, for reducing liver cirrhosis in the subject, for reducing liver granuloma formation in the subject, for reducing the number of hatched Schistosoma sp. parasites in the stool of the subject and/or for reducing IgGE sensitivity in the subject. In an embodiment, the subject is a mammal, such as, for example, a human. In still another embodiment, the Schistosoma sp. is Schistosoma mansoni.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the humoral response (e.g., the production of SmCB specific total IgG n=20) obtained with the various formulations tested. Serum from individual mice were analyzed by ELISA. Means and SEM are shown. NS=not significant, *P<0.05,**P<0.01***P<0.001.

FIG. 1A illustrates the production of SmCB specific IgG1 in immunized and challenged mice (n=20). Results are presented as antibody titers for PBS control mice (●), SmCB and Montanide (▪), SLA (▴), and AddaVax (▾).

FIG. 1B illustrates the production of SmCB specific IgG1 and IgG2c in immunized and challenged mice (n=10). Results are presented as the endpoint titer of SmCB specific IgG1 and IgG2c at week 9, in black and gray respectively.

FIG. 1C illustrates the production of SmCB specific IgE in immunized and challenged mice (n=10). Results are presented as antibody titers for PBS control mice (●), SmCB and Montanide (▪), SLA (▴), and AddaVax (▾).

FIG. 2 illustrates lymphoproliferation obtained with the various formulations tested. Splenocyte proliferation shown as stimulation index in response to SmCB restimulation ex vivo (N=10). Results are presented as the stimulation index for PBS control mice (●), SmCB and Montanide (▪), SLA (▴), and AddaVax (▾). Means are shown with SEM. Significance is calculated against the PBS control. *P<0.05, ***P<0.001.

FIG. 3 illustrates the cytokine and chemokine production obtained with the various formulations tested. Splenocyte supernatants (N=10) were run on a multiplex-ELISA for 16 cytokines and chemokines: IL-1α, IL-1β, IL-2, IL-3, IL-4, IL-5, IL-6, IL-10, IL-12, IL-17, MCP-1, IFNγ, TNFα, MIP-1α, RANTES, and GM-CSF. Significance is calculated against the PBS control. *P<0.05, **P<0.01, ***P<0.001, ****P<0.0001. Cytokines and chemokines have been grouped according to general functionality and labelled accordingly. Labels are colored reflecting the experimental group expressing the most amount of their cytokines/chemokines.

FIG. 3A shows the mean of cytokine/chemokine production along with standard deviation.

FIG. 3B shows the fold change above the PBS control group and depicted in the radar plot in with the axis in the natural log.

FIG. 4 illustrates the T cell response obtained with the various formulations tested. Splenocytes were restimulated with SmCB ex vivo and CD4⁺ and CD8⁺ T cells were assessed for their expression of IFNγ, IL-2, and TNF-α (N=10). Subtractive data is shown (stimulated cells—unstimulated cells). Significance is calculated against the PBS control. *P<0.05, **P<0.01, ***P<0.001.

FIG. 4A shows the response of CD4⁺ cells.

FIG. 4B shows the response of CD8⁺ cells.

FIG. 5 illustrates the parasitological outcomes obtained with the various formulations tested. Seven weeks after challenge, mice (N=10 per group) were euthanized, and worms and eggs were counted for parasite burden. Parasite burden reductions are shown as mean and SEM for adult worms, and hepatic eggs, and intestinal eggs, both adjusted per gram of tissue. Significance is calculated against the PBS control. *P<0.05, **P<0.01, ***P<0.001, ****P<0.0001.

FIG. 5A shows the parasite burden reductions for adult worms.

FIG. 5B shows the parasite burden for hepatic eggs.

FIG. 5C shows the parasite burden for intestinal eggs.

FIG. 6 illustrates the liver pathology obtained with the various formulations tested. Images of gross livers were taken, and liver sections were stained by H&E.

FIG. 6A shows images of gross livers. Representative liver images are shown for the PBS control on the left, and the experimental groups from left to right: Montanide, SLA, and AddaVax.

FIG. 6B shows liver sections stained by H&E. H&E staining of hepatic tissue shows an S. mansoni egg (pointed to with an arrow) within a granulomatous formation (within a black circle). H&E stained slides were viewed at 400×.

FIG. 7 illustrates the granuloma size and egg abnormality obtained with the various formulations tested. Significance was calculated against the PBS control. **P<0.01,***P<0.001, ****P<0.0001.

FIG. 7A shows the size, as determined using Zen Blue software, of 37-41 granulomas were measured per group of vaccinated animals and the mean and SEM of their size.

FIG. 7B shows, of the granulomas of FIG. 7A, when visualized in groups (15 groups of eggs were assessed per experimental group) a percentage of abnormal eggs was calculated and the mean and SEM of abnormality is shown in B.

FIG. 8 illustrates egg hatching obtained with the various formulations tested. Seven weeks after challenge, feces from mice was collected and hatched in water. The number of resulting miracidia was counted and adjusted to one gram of feces, and the mean and SEM are shown. Feces were collected from two independent mouse experiments, at two separate time points. Significance is calculated against the PBS control. *P<0.05.

FIG. 9 illustrates the gating strategy used for flow cytometry analysis. The same gating was done for each cell type CD4⁺ and CD8⁺ T cells and for each of those cell types the following cytokine expression was calculated: IFNγ, TNFα and IL-2. Subtractive data was used (Stimulated—unstimulated).

FIG. 10 illustrates the egg hatching set up used in the example. One gram of feces from each experimental group was resuspended in distilled water and placed into an Erlenmeyer flask/conical tube. The flask/tube was then wrapped in tin foil to protect from light and was topped up with distilled water so that about only 3 mm under the lid was exposed to light. Tin foil wrapped flasks were placed inside of a box, with a hole the same diameter as a lamp, and light was shone on them for three hours. After this time, water samples were collected from the exposed fraction of water and miracidia were counted.

FIG. 10A shows the Erlenmeyer flask/conical tube used in the egg hatching set up.

FIG. 10B shows the wrapped flask/tube used in the egg hatching set up.

FIG. 10C shows the box and light used in the egg hatching set up.

FIG. 11 illustrates the parasitological outcomes obtained with the various formulations tested. Seven weeks after challenge, mice (N=5 per group) were euthanized, and worms and eggs were counted for parasite burden. Parasite burden reductions are shown as mean and SEM for adult worms, and hepatic eggs, and intestinal eggs, both adjusted per gram of tissue. Significance is calculated against the PBS control. *P<0.05, **P<0.01, ***P<0.001, ****P<0.0001.

FIG. 11A shows the parasite burden reductions for adult worms.

FIG. 11B shows the parasite burden for hepatic eggs.

FIG. 11C shows the parasite burden for intestinal eggs.

FIG. 12A-B illustrates the construction of the AdSmCB and associated protein expression (determined using a Western blot).

FIG. 12A provides the genetic construct of the AdSmCB.

FIG. 12B provides Western blot of a cell lysate and a cell supernatant of Lane 1: Control, Lane 2: AdNeg, Lane 3: AdSmCB.

FIGS. 13A to 13F illustrates the specific humoral response using the various adenoviral preparations.

FIG. 13A provides the IgG titers (in ng/mL) in function of weeks of mice injected with PBS (●), SmCB (▪), Control Ad (Ad Neg SmCB ▴) and AdSmCB:SmCB (▾).

FIG. 13B provides the IgM titers (in ng/mL) in function of weeks of mice injected with PBS (●), SmCB (▪), Control Ad (Ad Neg SmCB ▴) and AdSmCB (▾).

FIG. 13C provides the IgE titers (in ng/mL) in function of weeks of mice injected with PBS (●), SmCB (▪), Control Ad (Ad Neg SmCB ▴) and AdSmCB (▾).

FIG. 13D provides the IgG avidity index of mice injected with SmCB (first column), Control Ad (second column) and AdSmCB:SmCB (third column).

FIG. 13E provides the endpoint IgG1 titers (Log 10) of mice injected with SmCB (first column), Control Ad (second column) and AdSmCB:SmCB (third column).

FIG. 13F provides the endpoint IgG2c titers (Log 10) of mice injected with SmCB (first column), Control Ad (second column) and AdSmCB:SmCB (third column).

FIGS. 14A and 14B illustrate the cytokine and chemokine production obtained with the various adenovirus tested.

FIG. 14A provides a radar plot comparing the levels of cytokines and chemokines obtained in splenocyte supernatants determined on a multiplex-ELISA. Labels are colored reflecting the experimental group expressing the most amount of their cytokines/chemokines.

FIG. 14B provides the IL-5 expression (pg/mL) in function of PBS (first column), SmCB (second column), Control Ad (AdNeg—third column) and AdSmCB:SmCB (fourth column).

FIG. 14C provides the INFγ expression (pg/mL) in function of PBS (first column), SmCB (second column), Control Ad (AdNeg—third column) and AdSmCB:SmCB (fourth column).

FIG. 14D provides the TNFα expression (pg/mL) in function of PBS (first column), SmCB (second column), Control Ad (AdNeg—third column) and AdSmCB:SmCB (fourth column).

FIG. 14E provides the RANTES expression (pg/mL) in function of PBS (first column), SmCB (second column), Control Ad (AdNeg—third column) and AdSmCB:SmCB (fourth column).

FIGS. 15A-E illustrate the T cell responses using different adenoviral preparations.

FIG. 15A provides the percentage of responding CD4+ cells in function of the cytokine expressed (INFγ, IL-2 or TNFα) as well as in function of PBS (first column), SmCB (second column), Control Ad (AdNeg—third column) and AdSmCB (fourth column).

FIG. 15B provides the percentage of responding CD8+ cells in function of the cytokine expressed (INFγ, IL-2 or TNFα) as well as in function of PBS (first column), SmCB (second column), Control Ad (AdNeg—third column) and AdSmCB (fourth column).

FIG. 15C provides the percentage of responding polyfunctional CD4+ cells in function of the number of cytokines expressed (1, 2 or 3) as well as in function of PBS (first column), SmCB (second column), Control Ad (AdNeg—third column) and AdSmCB (fourth column).

FIG. 15D provides the percentage of responding monofunctional and polyfunctional CD4+ cells in function of PBS (first column), SmCB (second column), Control Ad (AdNeg—third column) and AdSmCB (fourth column).

FIG. 15E provides the percentage of responding polyfunctional CD8+ cells in function of the number of cytokines expressed (1, 2 or 3) as well as in function of PBS (first column), SmCB (second column), Control Ad (AdNeg—third column) and AdSmCB (fourth column).

FIGS. 16A-C illustrate the parasitological outcomes obtained with the various adenoviral preparations tested.

FIG. 16A provides the percentage in worm reduction in function of PBS (first column), Control Ad (AdNeg—second column), SmCB (third column), Control Ad (AdNeg:SmCB—fourth column) and AdSmCB (fifth column).

FIG. 16B provides the percentage in hepatic egg reduction in function of PBS (first column), Control Ad (AdNeg—second column), SmCB (third column), Control Ad (AdNeg:SmCB—fourth column) and AdSmCB (fifth column).

FIG. 16C provides the percentage in intestinal egg reduction in function of PBS (first column), Control Ad (AdNeg—second column), SmCB (third column), Control Ad (AdNeg:SmCB—fourth column) and AdSmCB (fifth column).

FIGS. 17A-B illustrate the egg granuloma size and egg abnormality obtained with the various adenoviral preparations tested.

FIG. 17A provides the area (in μm²) of the granulomas observed in function of PBS, SmCB, Control Ad (AdNeg:SmCB) and AdSmCB.

FIG. 17B provides the percentage of abnormal eggs observed in function of PBS, SmCB, Control Ad (AdNeg:SmCB) and AdSmCB.

FIGS. 18A-B illustrates the CMI dose response obtained with the various adenoviral preparations tested.

FIG. 18A provides the percentage of CD4+ cells in function of the cytokine expressed (INFγ, IL-2 or TNFα) as well as iin function of PBS (first column), Control Ad (AdNeg—second column) and various doses of AdSmCB (10⁵, fourth column; 10⁷, fifth column; 10⁹ sixth column and 5×10⁹, seventh column).

FIG. 18B provides the percentage of CD8+ cells in function of the cytokine expressed (INFγ, IL-2 or TNFα) as well as iin function of PBS (first column), Control Ad (AdNeg—second column) and various doses of AdSmCB (10⁵, fourth column; 10⁷, fifth column; 10⁹ sixth column and 5×10⁹, seventh column).

FIGS. 19A-C illustrate the parasitological outcomes obtained with the various adenoviral preparations tested.

FIG. 19A provides the percentage in worm reduction in function of PBS (first column), Control Ad (AdNeg—second column), CatB (third column) and two doses of AdSmCB (10⁵, fourth column and 10⁹, fifth column).

FIG. 19B provides the percentage in hepatic egg reduction in function of PBS (first column), Control Ad (AdNeg—second column), CatB (third column) and two doses of AdSmCB (10⁵, fourth column and 10⁹, fifth column).

FIG. 19C provides the percentage in intestinal egg reduction in function of PBS (first column), Control Ad (AdNeg—second column), CatB (third column) and two doses of AdSmCB (10⁵, fourth column and 10⁹, fifth column).

FIGS. 20A-D characterizes of the cytokine and chemokine produced obtained with the various adenoviral preparations tested. Data are represented by means and SEM. Significance is calculated against the PBS control unless otherwise denoted. *P<0.05, **P<0.01, ***P<0.001.

FIG. 20A illustrates, from left to right, the IL1α, IL1β, IL2 and IL3 expression (pg/mL) of PBS (first column), SmCB (second column), Control Ad (AdNeg, third column) and AdSmCB (fourth column).

FIG. 20B illustrates, from left to right, the IL4, IL5, IL6 and IL10 expression (pg/mL) of PBS (first column), SmCB (second column), Control Ad (AdNeg, third column) and AdSmCB (fourth column).

FIG. 20C illustrates, from left to right, the IL12, IL17, MCP-1 and MP1α expression (pg/mL) of PBS (first column), SmCB (second column), Control Ad (AdNeg, third column) and AdSmCB (fourth column).

FIG. 20D illustrates, from left to right, the IFNγ, TNFα, RANTES and GMCSF expression (pg/mL) of PBS (first column), SmCB (second column), Control Ad (AdNeg, third column) and AdSmCB (fourth column).

### DETAILED DESCRIPTION

The present disclosure concerns an immunogenic composition, which can be a vaccine and comprises a Schistosoma epitope. In some embodiments, the composition is considered to be “immunogenic” because it is capable of eliciting a humoral response (e.g., the production of antibodies) against the Schistosoma epitope in a mammalian host or in vitro. For example, the composition can, in some embodiments, elicit the production of serum antibodies capable of binding to the Schistosoma epitope (and optionally opsonizing the infecting Schistosoma sp.). For example, the composition can, in some embodiments, elicit the production of IgG antibodies capable of binding to the Schistosoma epitope (such as, for example IgG1, IgG2, IgG3 and/or IgG4 antibodies and in particular IgG1 and/or IgG2c antibodies). In a specific example, the composition can, in some embodiments, elicit the production of a higher amount of IgG1 antibodies than IgG2c antibodies in a mammalian host or in vitro. In another example, the composition can, in some embodiments, elicit the production of IgA antibodies capable of binding to the Schistosoma epitope in a mammalian host or in vitro. In another example, the composition can, in some embodiments, elicit the production of IgM antibodies capable of binding to the Schistosoma epitope in a mammalian host or in vitro. In another example, the composition can, in some embodiments, elicit the production of IgE antibodies capable of binding to the Schistosoma epitope in a mammalian host or in vitro. In such embodiments, the composition preferably does not elicit IgE sensitivity in the mammalian host.

In some embodiments, the composition is considered to be “immunogenic” because it is capable of eliciting a Th2 (anti-inflammatory) response in a mammalian host or in vitro. In yet additional embodiments, the composition of the present disclosure increases the expression of interleukin(IL)-4 and/or IL-5. In some specific embodiments, the composition of the present disclosure can increase the expression of IL-4 by at least 1, 2, 3, 4, 5, 6, 7, 8, 9, 10 fold or more (when compared to a control composition lacking the Schistosoma sp. epitope like a phosphate-buffered saline or PBS). In some specific embodiments, the composition of the present disclosure can increase the expression of IL-5 by at least 100, 200, 300, 400, 500, 600, 700, 800, 900, 1 000 fold or more (when compared to a control composition lacking the Schistosoma sp. epitope like PBS).

In some embodiments, the composition is considered to be “immunogenic” because it is capable of eliciting a limited Th1 response in a mammalian host or in vitro. In yet additional embodiments, the composition of the present disclosure increases the expression of interferon(IFN) γ, IL-12 and/or tumor necrosis factor (TNF) α. In some specific embodiments, the composition of the present disclosure can increase the expression of INFγ by at least 10, 20, 30, 40, 50, 60, 70, 80, 90, 100 fold or more (when compared to a control composition lacking the Schistosoma sp. epitope like PBS). In some specific embodiments, the composition of the present disclosure can increase the expression of IL-12 by at least 1, 2, 3, 4, 5, 6, 7, 8, 9, 10 fold or more (when compared to a control composition lacking the Schistosoma sp. epitope like PBS). In some specific embodiments, the composition of the present disclosure can increase the expression of TNFα by at least 1, 2, 3, 4, 5, 6, 7, 8, 9, 10 fold or more (when compared to a control composition lacking the Schistosoma sp. epitope like PBS).

In some embodiments, the composition is considered to be “immunogenic” because it is capable of eliciting a limited inflammatory response in a mammalian host or in vitro. In yet additional embodiments, the composition of the present disclosure does not substantially increase the expression of IL-1α (when compared to a control composition lacking the Schistosoma sp. epitope like PBS). In yet additional embodiments, the composition of the present disclosure increases the expression of IL-1β, IL-6, monocyte chemoattractant protein-1 (MCP-1), macrophage inflammatory protein-1α (MIP-1α), granulocyte monocyte colony stimulating factor (GM-CSF) and/or TNFα (when compared to a control composition lacking the Schistosoma sp. epitope like PBS). In some specific embodiments, the composition of the present disclosure can increase the expression of IL-1β by at least 1, 2, 3, 4, 5, 6, 7, 8, 9, 10 fold or more (when compared to a control composition lacking the Schistosoma sp. epitope like PBS). In some specific embodiments, the composition of the present disclosure can increase the expression of IL-6 by at least 10, 20, 30, 40, 50, 60, 70, 80, 90, 100 fold or more (when compared to a control composition lacking the Schistosoma sp. epitope like PBS). In some specific embodiments, the composition of the present disclosure can increase the expression of MCP-1 by at least 10, 20, 30, 40, 50, 60, 70, 80, 90, 100 fold or more (when compared to a control composition lacking the Schistosoma sp. epitope like PBS). In some specific embodiments, the composition of the present disclosure can increase the expression of MIP-1a by at least 10, 20, 30, 40, 50, 60, 70, 80, 90, 100 fold or more (when compared to a control composition lacking the Schistosoma sp. epitope like PBS). In some specific embodiments, the composition of the present disclosure can increase the expression of GM-CSF by at least 1, 2, 3, 4, 5, 6, 7, 8, 9, 10 fold or more (when compared to a control composition lacking the Schistosoma sp. epitope like PBS). In some specific embodiments, the composition of the present disclosure can increase the expression of TNFα by at least 1, 2, 3, 4, 5, 6, 7, 8, 9, 10 fold or more (when compared to a control composition lacking the Schistosoma sp. epitope like PBS).

In some embodiments, the composition is considered to be “immunogenic” because it is capable of eliciting an anti-inflammatory response in a mammalian host or in vitro. In yet additional embodiments, the composition of the present disclosure increases the expression of IL-10 (when compared to a control composition lacking the Schistosoma sp. epitope like PBS). In some specific embodiments, the composition of the present disclosure can increase the expression of IL-10 by at least 1, 2, 3, 4, 5, 6, 7, 8, 9, 10 fold or more (when compared to a control composition lacking the Schistosoma sp. epitope like PBS).

In some embodiments, the composition is considered to be “immunogenic” because it is capable of eliciting a myeloid cell proliferation response in a mammalian host or in vitro. In yet additional embodiments, the composition of the present disclosure increases the expression of IL-3 (when compared to a control composition lacking the Schistosoma sp. epitope like PBS). In some specific embodiments, the composition of the present disclosure can increase the expression of IL-3 by at least 1, 2, 3, 4, 5, 6, 7, 8, 9, 10 fold or more (when compared to a control composition lacking the Schistosoma sp. epitope like PBS).

In some embodiments, the composition is considered to be “immunogenic” because it is capable of eliciting a limited T cell associated response in a mammalian host or in vitro. In yet additional embodiments, the composition of the present disclosure increases the expression of IL-2 and/or RANTES or CCL-5 regulated upon activation, normal T cell expressed and secreted (when compared to a control composition lacking the Schistosoma sp. epitope like PBS). In some specific embodiments, the composition of the present disclosure can increase the expression of IL-2 by at least 1, 2, 3, 4, 5, 6, 7, 8, 9, 10 fold or more (when compared to a control composition lacking the Schistosoma sp. epitope like PBS). In some specific embodiments, the composition of the present disclosure can increase the expression of RANTES by at least 1, 2, 3, 4, 5, 6, 7, 8, 9, 10 fold or more (when compared to a control composition lacking the Schistosoma sp. epitope like PBS).

In some embodiments, the composition is considered to be “immunogenic” because it is capable of eliciting a limited Th17 response in a mammalian host or in vitro. In yet additional embodiments, the composition of the present disclosure increases the expression of IL-17 (when compared to a control composition lacking the Schistosoma sp. epitope like PBS). In some specific embodiments, the composition of the present disclosure can increase the expression of IL-17 by at least 1, 2, 3, 4, 5, 6, 7, 8, 9, 10 fold or more (when compared to a control composition lacking the Schistosoma sp. epitope like PBS).

The immunogenic composition of the present disclosure includes a Schistosoma epitope. The epitope is derived from a peptide or a protein being expressed by a Schistosoma sp. For example, the epitope may be derived from Schistosoma japonicum, Schistosoma mansoni, Schistosoma bovis, Schistosoma heamatobium, Schistosoma intercalatum, Schistosoma guineensis, Schistosoma curassoni, Schistosoma mattheei or Schistosoma mekongi. In a specific embodiment, the epitope is derived from a protein expressed by Schistosoma mansoni. The epitope or the protein/peptide bearing such epitope may be glycosylated. The epitope or the protein/peptide bearing such epitope may be modified to as to add and/or remove one or more putative glycosylation site. The epitope may be present on a full length protein expressed by a Schistosoma sp., a variant of the full length protein expressed by a Schistosoma sp. or on a fragment of the native or variant protein expressed by a Schistosoma sp. It is contemplated that the immunogenic composition comprises a single epitope or a plurality of epitopes. When the immunogenic composition comprises a single epitope, the epitope can be located on a single polypeptide (or polypeptide fragment) or on different polypeptides (or polypeptide fragments). When the composition comprises more than one different epitope, each epitope can be provide on a single polypeptide (or polypeptide fragment) or on different polypeptides (or polypeptide fragments). The Schistosoma sp. protein/variant/fragment comprising the epitope may be expressed in one or more steps of the worm's life cycle: it may be expressed in at the egg stage, at the miracidia stage, at the sprorocyst stage, at the cercariae stage and/or at the adult worm stage. The Schistosoma sp. protein can be purified from any step of the worm's life cycle or can be recombinantly expressed in an heterologous host.

The amount of Schistosoma sp. protein present in each dose of the immunogenic composition can be between about 1 to 1000 μg. In an embodiment, the amount of Schistosoma sp. protein present in each dose of the immunogenic composition can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 100, 200, 300, 400, 500, 600, 700, 800, 900, 1000 μg or more. In an embodiment, the amount of Schistosoma sp. protein present in each dose of the immunogenic composition can be below 1000, 900, 800, 700, 600, 500, 400, 300, 200, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 pg or less. In an embodiment, the amount of Schistosoma sp. protein present in each dose of the immunogenic composition is about 10 μg. In an embodiment, the amount of Schistosoma sp. protein present in each dose of the immunogenic composition is about 20 μg. In an embodiment, the amount of Schistosoma sp. protein present in each dose of the immunogenic composition is about 30 μg. In an embodiment, the amount of Schistosoma sp. protein present in each dose of the immunogenic composition is about 100 μg.

In some embodiments, the Schistosoma sp. epitope is located on the Sm-p80, Sm29, G3PDH, Sm-TSP2 or cathepsin B protein (also referred to as the Sm31 antigen). In one embodiment, the Schistosoma sp. epitope is located on the cathepsin B protein (also referred to as the Sm31 antigen), a variant of the cathepsin B protein or a fragment of the cathepsin B protein. Cathepsin B is the most abundant cysteine protease found in schistosomula and adult worm gut and somatic extracts. In some embodiments, the epitope is derived from the cathepsin B protein of S. mansoni (GenBanK accession number AAA29865), S japonicum (GenBanK accession number CAA50305), S. mekongi (GenBanK accession number AKU37272), S. bovis (GenBanK accession number RTG87606) or S. haematobium (GenBanK accession number XP_012801036). In an embodiment, the Schistosoma sp. protein comprising the epitope can be a full-length version of the cathepsin B protein (which may, in some embodiments, exclude its signal sequence), a variant thereof or a fragment thereof.

As used in the context of the present disclosure, a “variant” comprises at least one amino acid difference (substitution or addition) when compared to, for example, the amino acid sequence of the native cathepsin B and still possesses the one or more epitope present on the native cathepsin B. The variant does not need to exhibit the biological activity associated with the native cathepsin B protein as long as it retains the immunogenic properties of the native cathepsin B protein or has an increase immunogenic properties. In an embodiment, the variant peptide or polypeptide exhibits at least 50%, 60%, 70%, 80%, 90%, 95%, 96%, 97%, 98% or 99% of the immunogenic property of native cathepsin B protein. The variants also have at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to the native cathepsin B protein. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5. In an embodiment, the variant cathepsin B protein is a protein in which a putative glycosylation site has been removed. Known immunogenic cathepsin B fragments include, but are not limited to, fragments described in Noya et al. (2001). In a specific embodiment, the cathepsin B protein corresponds to the S. mansoni cathepsin B protein in which a putative glycosylation site has been removed. In yet a further specific embodiment, the cathepsin B protein corresponds to the S. mansoni cathepsin B (GenBanK accession number AAA29865) at which, at position 183, the asparagine residue has been replaced with a glycine residue.

The present disclosure also provide fragments of the native cathepsin B protein and variants associated thereto. A fragment comprises at least one less amino acid residue when compared to the amino acid sequence of the native cathepsin B protein or variant and still possess at least the same or increased immunogenic properties when compared to the native cathepsin B or variant thereof. In an embodiment, the fragment peptide or polypeptide exhibits at least 50%, 60%, 70%, 80%, 90%, 95%, 96%, 97%, 98% or 99% of the immunogenic properties of native cathepsin B protein or variant thereof. The fragment peptide or polypeptide can also have at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to the native cathepsin B protein or variant thereof. The fragment can be, for example, a truncation of one or more amino acid residues at the amino-terminus, the carboxy terminus or both terminus of the native heterologous peptide, polypeptide or fragment thereof. Alternatively or in combination, the fragment can be generated from removing one or more internal amino acid residues. In an embodiment, the fragment peptide or polypeptide has at least 5, 6, 7, 8, 9, 10, 11, 12, 13, 14, 15, 20, 40, 60, 80, 100, 150, 200, 250, 300, 350, 400 or more consecutive amino acids of the native cathepsin B protein or the variant thereof. In a specific embodiment, the cathepsin B protein corresponds to the S. mansoni cathepsin B protein in which the signal peptide has been removed. In yet a further specific embodiment, the cathepsin B protein corresponds to the S. mansoni cathepsin B (GenBanK accession number AAA29865) lacking the first 17 amino acid residues (which correspond to the signal peptide). In yet a further specific embodiment, the cathepsin B protein corresponds to the S. mansoni cathepsin B (GenBanK accession number AAA29865) lacking the first 17 amino acid residues and at which, at position 183, the asparagine residue has been replaced with a glycine residue.

The peptide or polypeptide comprising the Schistosoma epitope can be chemically modified so as to increase its persistence in a tissue or the circulation. For example, the peptide or protein comprising the Schistosoma epitope can be conjugated to a lipid, a polyethylene glycol molecule, a steroid, a saccharide, a carrier protein (such as albumin for example) or polyamine for stabilization. If the peptide or polypeptide comprising the Schistosoma epitope is chemically modified, care must be taken so as to preserve its immunogenic properties and stability in the composition. In some embodiments, the peptide or protein comprising the Schistosoma epitope can be provided as a pharmaceutically acceptable salt form.

The immunogenic composition of the present disclosure can comprise an emulsion of a Schistosoma epitope. As it is known in the art, an emulsion is a mixture of at least two distinct liquid phases (e.g., a continuous phase and a dispersed phase) which are immiscible. In one embodiment, the emulsion is an oil-in-water emulsion, e.g., an emulsion in which the water phase forms the continuous phase and the oil phase is dispersed (as droplets or particles) in within the water phase. Alternatively or in combination, the emulsion can be a nanoemulsion, e.g., an emulsion in which the dispersed phase forms droplets/particles in the nanometer range. In some embodiments, the droplets/particles have a diameter size of less than 1000 nM, 500 nM, 400 nM, 300 nM, 200 nM, 100 nM or below. In some specific embodiments, the droplets/particles of the nanoemulsion have a diameter size between 100 and 200 nM. For example, the droplets/particles of the nanoemulsion have a diameter size of at least 100, 110, 120, 130, 140, 150, 160, 170, 180 or 190 nM and no more than 200, 190, 180, 170, 160, 150, 140, 130, 120 or 100 nM. In a further example, the droplets/particles of the nanoemulsion have a diameter size of about 160 nM. In some embodiments, the emulsion is an oil-in-water nanoemulsion.

The oil phase of the emulsion of the immunogenic composition can include a squalene (PubChem ID 638072) or a squalene derivative. The squalene or the squalene derivative can serve as an adjuvant in the immunogenic composition. In a single dose of the immunogenic composition of the present disclosure, the squalene oil phase can be present at a concentration between about 1 and 10 volume/volume (based on the total volume of the immunogenic composition). In an embodiment, the single dose of the immunogenic composition can comprise at least 1, 2, 3, 4, 5, 6, 7, 8, 9 or 10 v/v of squalene oil. In other embodiment, the single dose of the immunogenic composition can comprise no more than 10, 9, 8, 7, 6, 5, 4, 3, 2 or 1 v/v of squalene oil. In yet a further embodiment, the single dose of the immunogenic composition can comprise about 5% v/v of the squalene oil.

In some embodiments, the immunogenic compositions of the present disclosure can include an emulsifier. Emulsifier materials concentrate at the phase interface (between the oil phase and the water phase) to lower the interfacial tension. Emulsifiers are understood to reduce the energy required to break the dispersed oil phase into droplets and prevent them from coalescing by generating a repulsive force or a physical barrier between them. Emulsifiers preferably locate in the oil phase of the emulsion. Emulsifying agents can be classified according to their chemical structure or mechanism of action. Classes according to chemical structure are synthetic, natural, finely dispersed solids, and auxiliary agents. Classes according to mechanism of action are monomolecular, multimolecular, and solid particle films. Regardless of their classification, emulsifiers must preferably be chemically stable in the system, inert and chemically non-reactive with other emulsion components, and nontoxic and non-irritant. Some commonly used emulsifiers include tragacanth, sodium lauryl sulfate, sodium dioctyl sulfosuccinate, and polymers known as the Spans® (sorbitol esters) and Tweens®. In an embodiment, the emulsifier is a fatty acid ester, such as, for example a sorbitan fatty acid ester. In yet a further embodiment, the emulsifier can be sorbitan trioleate. In some embodiments, a single dose of the immunogenic composition of the present disclosure can include between 0.01% and 10% (weight per volume of the immunogenic composition) of the emulsifier. In a specific embodiment, the single dose of the immunogenic composition comprises at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10% (weight per volume of the immunogenic composition) of the emulsifier. In another specific embodiment, the single dose of the immunogenic composition comprises no more than 10.0, 9.0, 8.0, 7.0, 6.0, 5.0, 4.0, 3.0, 2.0, 1.0, 0.9, 0.8, 0.7, 0.6, 0.5, 0.4, 0.3, 0.2, 0.1, 0.09, 0.08, 0.07, 0.06, 0.05, 0.04, 0.03, 0.02 or 0.01% (weight per volume of the immunogenic composition) of the emulsifier. In yet another embodiment, the single dose of the immunogenic composition of the present disclosure comprises about 0.5% (weight per volume of the immunogenic composition) of the emulsifier.

The water phase of the emulsion present in the immunogenic composition of the present disclosure comprises the Schistosoma epitope. The peptide or the polypeptide comprising the Schistosoma epitope is at least in part or totally present in the water phase of the emulsion.

The water phase of the emulsion present in the immunogenic composition of the present disclosure can include a buffering system so as to maintain the pH of the composition during the preparation and the storage of the composition. The buffer is a physiologically acceptable buffer which as a pKa between about 6.0 and 8.0 and is preferably soluble in the water phase, but not substantially soluble in the oil phase. The buffer of the immunogenic compositions of the present disclosure can be, for example, a phosphate buffer, a citrate buffer, a bicarbonate buffer, an acetate buffer as well as combinations thereof. In an embodiment, the buffer is a citrate buffer, such as, for example a sodium citrate buffer.

In some embodiments, the immunogenic compositions of the present disclosure can include a surfactant. Surfactants concentrate at the phase interface (between the oil phase and the water phase) to lower the interfacial tension. Like emulsifiers, surfactants are understood to reduce the energy required to break the dispersed oil phase into droplets and prevent them from coalescing by generating a repulsive force or a physical barrier between them. Surfactants preferably locate in the water phase of the emulsion. Surfactants can be classified according to their chemical structure or mechanism of action. Classes according to chemical structure are synthetic, natural, finely dispersed solids, and auxiliary agents. Classes according to mechanism of action are monomolecular, multimolecular, and solid particle films. Regardless of their classification, surfactants must preferably be chemically stable in the system, inert and chemically non-reactive with other emulsion components, and nontoxic and non-irritant. Some commonly used surfactants include tragacanth, sodium lauryl sulfate, sodium dioctyl sulfosuccinate, and polymers known as the Spans® (sorbitol esters) and Tweens® (polysorbates). In an embodiment, the surfactant is a non-ionic surfactant. In still a further embodiment, the surfactant can be a polysorbate, such as, for example, polysorbate 80. In some embodiments, the immunogenic composition of the present disclosure can include between 0.01% and 10% (weight per volume of the immunogenic composition) of the surfactant. In a specific embodiment, the immunogenic composition comprises at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10% (weight per volume of the immunogenic composition) of the surfactant. In another specific embodiment, the immunogenic composition comprises no more than 10.0, 9.0, 8.0, 7.0, 6.0, 5.0, 4.0, 3.0, 2.0, 1.0, 0.9, 0.8, 0.7, 0.6, 0.5, 0.4, 0.3, 0.2, 0.1, 0.09, 0.08, 0.07, 0.06, 0.05, 0.04, 0.03, 0.02 or 0.01% (weight per volume of the immunogenic composition) of the surfactant. In yet another embodiment, the immunogenic composition of the present disclosure comprises about 0.5% (weight per volume of the immunogenic composition) of the surfactant.

In additional embodiments, the immunogenic composition of the present disclosure can comprising a nucleic acid molecule encoding cathepsin B, an immunogenic cathepsin B variant or an immunogenic cathepsin B fragment. The nucleic acid molecule of the composition in not, in its intact nature immunogenic per se, but it is intended to be capable of being expressing upon administration to the host and thereby allowing the production of cathepsin B, the immunogenic cathepsin B variant or the immunogenic cathepsin B fragment in the host. The cathepsin B, the immunogenic cathepsin B variant or the immunogenic cathepsin B fragment being expressed in the host is immunogenic and thus capable of eliciting a cellular and/or a humoral response. The nucleic acid molecule can, in some embodiments, comprise ribonucleic acid residues, deoxyribonucleic acid residues or a combination of both ribonucleic acid residues and deoxyribonucleic acid residues. In some embodiment, the nucleic molecule is located in a viral-derived vector, such as, for example, an adenovirus-derived vector. A “viral-derived” vector is a nucleic acid molecule which has been derived in part from a virus and which allows the expression of the polypeptides encoded by the nucleic acid molecule. The viral-derived vector may, in some embodiments, be a replication-deficient viral-derived vector. In embodiments in which the viral-derived vector is an adenovirus-derived vector, the replication-deficient adenovirus-derived vector may lack E1 and/or E3. The immunogenic composition can include one or more additional polypeptides, for example, polypeptides encoded by the viral-derived vector. In some embodiments, the immunogenic composition can include or be used in combination with a source of a polypeptide comprising cathepsin B, the immunogenic cathepsin B variant or the immunogenic cathepsin B fragment. In some embodiments, the source of a polypeptide comprising cathepsin B, the immunogenic cathepsin B variant or the immunogenic cathepsin B fragment can be provided as the emulsion described herewith.

The immunogenic composition can include additional components such as, for example, a further adjuvant, a surfactant, a salt, a bulking agent, one or more preservative (including one or more antibiotic). Such additional components must be suitable to be incorporated in the emulsion, either in the water phase, the oil phase or at the interphase of both phases.

The immunogenic composition can be provided as a concentrated liquid to be diluted prior to administration to the subject. The immunogenic composition can be provided in a liquid form ready to be administrated to the subject. The immunogenic composition can be provided in a solid (powder) form to be diluted/resuspended with water or an aqueous solution prior to administration to the subject. The immunogenic composition can be provided in a vial for a single or multiple uses. The immunogenic composition can be provided in a pre-filled syringe to a single or multiple uses.

The present disclosure also provides a pharmaceutical composition comprising the immunogenic composition with an excipient or a carrier. In accordance with the present invention, an excipient or a carrier is a pharmaceutically acceptable solvent, suspending agent or any other pharmacologically inert vehicle for delivering one or more Schistosoma epitope to a subject. The carrier is typically liquid or solid (intended to be reconstituted prior to the administration to the subject). A pharmaceutical carrier/excipient is generally selected to provide for the desired bulk, consistency, etc., when combined with components of a given pharmaceutical composition, in view of the intended administration mode. The pharmaceutically acceptable carriers or excipients may be solvents, vehicles or medium. The medium can be for example saline, buffered saline, dextrose, water, glycerol, ethanol, propylene glycol, or polyethylene glycol.

The present disclosure also provides the immunogenic composition in the form of a vaccine. As it is known in the art, a vaccine is a pharmaceutical composition intended to be administrated to a subject in order to prevent, reduce the severity, reduce the infectious burden and/or treat an infection.

In an embodiment, the pharmaceutical composition or the vaccine of the present disclosure is adapted for delivery by at least one route consisting of dermal, transdermal, intravenous, intraarterial, parenteral, subcutaneous, intramuscular, intracranial, intraorbital, ophthalmic, intraventricular, intracapsular, intraspinal, intrathecal, epidural, intracisternal, pulmonary, intraperitoneal, rectal, buccal (including sublingual), and intranasal. In a specific embodiment, the immune composition/pharmaceutical composition/vaccine of the present disclosure are formulated for an intramuscular administration.

The present disclosure also provides a process for making an embodiment of the immunogenic composition, the pharmaceutical composition as well as the vaccine of the present disclosure. The process comprises providing or making an emulsion as described herein. As such, the process can include admixing the water phase and the oil phase so as to obtain the emulsion. The process can also including supplementing the oil phase with an emulsifier and/or the water phase with a buffering system and/or a surfactant before admixing both phases. When the emulsion is a nanoemulsion, the process can use a microfluidizer to combine the water and the oil phase and create the emulsion. Optionally, the process can also filter the nanoemulsion (in a 0.22 μM filter for example) to select for a preferred droplet/particle size. Once the emulsion has been made, the Schistosoma epitope (which may be provided dried or in a concentration form in a saline or buffer solution for example) can be diluted in same to provide the immunogenic composition. The resulting composition can be filtered, dried (at least in part) and dispenses into containers prior to use.

When the immunogenic composition is provided as a pharmaceutical composition, the process can include admixing the emulsion or the immunogenic composition with the pharmaceutical carrier/excipient prior to use. When the immunogenic composition is provided as a vaccine, the process can include drying the immunogenic composition to provide a solid (e.g., a resuspendable powder for example) and/or dispensing the immunogenic composition in a device for administering the vaccine (e.g., a pre-filled syringe for example).

The immunogenic compositions, pharmaceutical compositions and vaccines of the present disclosure can be used for the prevention, the reduction of the severity and/or the treatment of a Schistosoma sp. infection in a subject in need thereof. In an embodiment, the subject can be a mammal, for example, a human. The infection can be caused, for example, Schistosoma japonicum, Schistosoma mansoni, Schistosoma bovis, Schistosoma heamatobium, Schistosoma intercalatum, Schistosoma guineensis, Schistosoma curassoni, Schistosoma hmattheei or Schistosoma mekongi. In a specific embodiment, the Schistosoma infection can be or is caused by Schistosoma mansoni.

The immunogenic compositions, pharmaceutical compositions and vaccines of the present disclosure can be used for reducing one or more symptoms associated with a Schistosoma sp. infection in a subject. Symptoms associated with a Schistosoma sp. infection include, without limitations, during the initial phase: a fever and/or a rash, and, in the chronic phase: a diarrhea, a cough, muscle and/or joint pain, etc.

The immunogenic compositions, pharmaceutical compositions and vaccines of the present disclosure can be used to prevent the onset of a Schistosoma sp. infection in a subject, to eradicate a Schistosoma sp. infection in an infected subject, to reduce the number of adult Schistosoma sp. worms/eggs in an infected subject, to reduce liver cirrhosis in an infected subject, to reduce liver granuloma formation in an infected subject, to reduce the number of hatched Schistosoma sp. parasites in the stool of an infected subject and/or to reduce IgGE sensitivity in the infected subject. The method can include, prior to the administration of the immunogenic compositions, pharmaceutical compositions and vaccines of the present disclosure, determining if the subject is infected by a Schistosoma sp., the presence (and optionally the number) of worms/eggs in the subject, the presence (and optionally the severity) of liver cirrhosis in the subject, the presence (and optionally the severity) of liver granuloma formation in an infected subject, the presence (and optionally the number) of hatched Schistosoma sp. parasites in the stool of the subject and/or the presence (and optionally the severity) of IgGE sensitivity in the subject. Alternatively or in combination, the method can include, after the administration of at least one dose of the immunogenic compositions, pharmaceutical compositions and vaccines of the present disclosure, determining if the subject is infected by a Schistosoma sp., the presence (and optionally the number) of worms/eggs in the subject, the presence (and optionally the severity) of liver cirrhosis in the subject, the presence (and optionally the severity) of liver granuloma formation in an infected subject, the presence (and optionally the number) of hatched Schistosoma sp. parasites in the stool of the subject and/or the presence (and optionally the severity) of IgGE sensitivity in the subject. Such determinations can be made for example to assess if more than one dose of the immunogenic composition is necessary to achieve the desired therapeutic dose.

The immunogenic compositions, pharmaceutical compositions and vaccines of the present disclosure can be used for the prevention, the reduction of the severity and/or the treatment of a Schistosoma sp. infection in a subject in need thereof. In an embodiment, the subject can be a mammal, for example, a human. The infection can be caused, for example, Schistosoma japonicum, Schistosoma mansoni, Schistosoma bovis, Schistosoma heamatobium, Schistosoma intercalatum, Schistosoma guineensis, Schistosoma curassoni, Schistosoma hmattheei or Schistosoma mekongi. In a specific embodiment, the Schistosoma infection can be or is caused by Schistosoma mansoni.

The immunogenic compositions, pharmaceutical compositions and vaccines of the present disclosure can be used to provide or increase the humoral (for example IgG production such as IgG1 production) or cellular immunity against Schistosoma sp. in a subject and/or to favor a Th2 immune response (e.g., cytokines/chemokines associated with the Th2 response) against a Schistosoma sp. infection in a subject. The method can include, prior to the administration of the immunogenic compositions, pharmaceutical compositions and vaccines of the present disclosure, determining if the subject exhibits a pre-existing humoral or cellular immunity against Schistosoma sp. in a subject and/or a Th2 immune response against Schistosoma sp. The method can include, after the administration of at least one dose the immunogenic compositions, pharmaceutical compositions and vaccines of the present disclosure, determining if the subject exhibits a humoral or cellular immunity against Schistosoma sp. in a subject and/or a Th2 immune response against Schistosoma sp. Such determinations can be made for example to assess if more than one dose of the immunogenic composition is necessary to achieve the desired therapeutic goal.

As shown in the Examples below, the immunogenic compositions, pharmaceutical compositions and vaccines of the present disclosure can be used to stimulate, ex vivo, the immune response of leukocytes (splenocytes when the subject is a mouse).

In the context of the present disclosure, it is contemplated that the immunogenic compositions, the pharmaceutical compositions and the vaccines of the present disclosure be administered to a subject which has been previously diagnosed with a Schistosoma sp. infection. Diagnostic methods which can be used are known in the art and include, without limitations the determination of the presence and/or concentration of Schistosoma sp. eggs, antibodies or antigens in stool or urine.

The immunogenic compositions, the pharmaceutical compositions and the vaccines of the present disclosure can be administered as a single dose or can be administered in multiple doses. The first dose is usually referred as a priming dose and can, in some embodiments, be sufficient to achieve the therapeutic goal. In some alternative embodiments, more than one dose of the immunogenic compositions, the pharmaceutical compositions and the vaccines can and should be administered to achieve the therapeutic goal. The subsequent doses are usually referred to as booster or maintenance doses. The priming and the booster doses can be administered at an interval of one or more days, one or more months or one or more years. In an embodiment, the pharmaceutical compositions and the vaccines of the present disclosure are administered as a first priming dose and subsequently, at a later point in time, as a second booster dose. In some additional embodiments, the pharmaceutical compositions and the vaccines of the present disclosure are administered as a first priming dose and subsequently, at a later point in time, as a second and a third booster doses.

In embodiments in which the immunogenic composition comprises the nucleic acid molecule encoding the cathepsin B, the immunogenic cathepsin B variant or the immunogenic cathepsin B fragment and is used in combination with a further source of a polypeptide comprising cathepsin B, the immunogenic cathepsin B variant or the immunogenic cathepsin B fragment, the nucleic acid molecule can be administered as a first dose and the further source of the polypeptide can be administered as one or more further booster doses. Alternatively, the further source of the polypeptide can be administered as a first dose and the composition comprising the nucleic acid molecule as one or more further booster doses. In some instances, the further source of the polypeptide is administered alternatively with the composition comprising the nucleic acid molecule. In additional instances, the composition comprising the nucleic acid molecule is administered alternatively with the further source of the polypeptide.

The immunogenic compositions, the pharmaceutical compositions and the vaccines of the present disclosure can be used alone or in combination with other therapeutics used for the treatment of a Schistosoma sp. infection. For example, the immunogenic compositions, the pharmaceutical compositions and the vaccines of the present disclosure can be used in combination with one or more anthelminthic drug such as, for example, praziquantel and/or oxaminiquine.

The immunogenic compositions, the pharmaceutical compositions and the vaccines of the present disclosure include an effective amount of the Schistosoma sp. epitope. As used in the context of the present disclosure, the terms “effective amount”, “pharmaceutically effective amount” or “therapeutically effective amount” refers to an amount (dose) effective to achieve the therapeutic goal in the subject. It is also to be understood herein that an “effective amount” may be interpreted as an amount giving a desired therapeutic effect, either taken in one dose or in any dosage or route, taken alone or in combination with other therapeutic agents. The dose of the Schistosoma sp. epitope depends on a number of factors, such as, e.g., the manner of administration, the age and the body weight of the subject, and the condition of the subject to be treated, and ultimately will be decided by the attending physician or veterinarian. Such an amount of the Schistosoma sp. epitope as determined by the attending physician or veterinarian is referred to herein, and in the claims, as an “effective amount”.

The present invention will be more readily understood by referring to the following examples which are given to illustrate the invention rather than to limit its scope.

### EXAMPLE I

Immunization Protocol. Six to eight-week old female C57BL/6 mice were bred from mice obtained from Charles River Laboratories (Senneville, QC). Eight groups of mice (n=5/gp) were immunized for parasite burden reduction. Group 1: control: mice were injected with phosphate-buffered saline (PBS) (Wisent Bioproducts, St. Bruno, QC). Group 2: positive control: mice were immunized with 20 μg of recombinant SmCB (rSmCB) and 35 μL of Montanide ISA 720 VG (SEPPIC Inc., Fairfield, NJ). Group 3: mice were immunized with 20 μg rSm-CB. Group 4: mice were immunized with 20 μg rSm-CB and 25 μL of AddaVax™ (InvivoGen, San Diego, CA). Group 5: mice were immunized with 20 μg rSm-CB and 40 μg of aluminum hydroxide (alum; Alhydrogel; Brenntag BioSector A/S, Frederikssund, Denmark). Group 6: mice were immunized with 20 μg rSm-CB and 40 μg of aluminum hydroxide and 10 μg CpG dinucleotides (Hycult Biotechnology B.V., Netherlands) Group 7: mice were immunized with 20 μg rSmCB admixed with 1 mg of pre-formed empty SLA archaeosomes (NRC, Ottawa, Canada). Each mouse was immunized at weeks 0, 3, and 6 intramuscularly in the thigh with 50 μL of vaccine. Group 8: mice were immunized with 20 μg rSm-CB and 40 μg of aluminum hydroxide and 10 μg monophosphoryl lipid A (List Biological Laboratories, California).

Schistosoma mansoni challenge. Biomphalaria glabrata snails infected with the Puerto Rican strain of S. mansoni were provided by NIAID Schistosomiasis Resource Center of the Biomedical Research Institute (Rockville, MD). Three weeks after the final immunization mice were challenged with 150 cercaria via tail exposure and sacrificed seven weeks later. Images of mouse livers were taken during dissection using a Galaxy S8 cell phone camera (Samsung Group, Seoul, South Korea). Adult worms were perfused from the hepatic portal system and counted manually (Ricciardi et al., 2016).

Upon infection, all vaccine groups were able to reduce parasite burden greater than 40%. Adult worms were reduced by 79%, 50%, 95%, 62%, 65%, 68%, and 69% in the groups Montanide, antigen alone, AddaVax, Aluminum hydroxide (alum), alum and CpG, SLA, and alum and MPL respectively. The same groups reduced liver eggs by 75%, 52%, 88%, 35%, 55%, 59%, and 79% respectively. And the same groups also reduced intestinal eggs by 78%, 55%, 95%, 43%, 78%, 77%, and 77% respectively. These parasite burden reductions are significant compared to the WHO threshold of 40%, and continue to support the hypothesis that Cathepsin B is a good candidate for vaccine formulations.

Ethics statement. All animal procedures were performed in accordance with Institutional Animal Care and Use Guidelines approved by the Animal Care and Use Committee at McGill University (Animal Use Protocol 7625).

Sm-Cathepsin B Recombinant Protein Preparation. S. mansoni Cathepsin B was prepared and purified as we previously described (Ricciardi et al., 2015).

Immunization Protocol. Six to eight-week old female C57BL/6 mice were bred from mice obtained from Charles River Laboratories (Senneville, QC). Four groups of mice (n=10/gp) were immunized for humoral and cytokine assessment. Four groups of mice, (n=10/gp) were immunized and subsequently infected for parasite burden assessment. Group 1: control: mice were injected with phosphate-buffered saline (PBS) (Wisent Bioproducts, St. Bruno, QC). Group 2: positive control: mice were immunized with 20 μg of recombinant SmCB (rSmCB) and 35 μL of Montanide ISA 720 VG (SEPPIC Inc., Fairfield, NJ). Group 3: mice were immunized with 20 μg rSmCB admixed with 1 mg of pre-formed empty SLA archaeosomes (NRC, Ottawa, Canada). Group 4: mice were immunized with 20 μg rSm-CB and 25 μL of AddaVax™ (InvivoGen, San Diego, CA). Each mouse was immunized at weeks 0, 3, and 6 intramuscularly in the thigh with 50 μL of vaccine.

Schistosoma mansoni challenge. Biomphalaria glabrata snails infected with the Puerto Rican strain of S. mansoni were provided by NIAID Schistosomiasis Resource Center of the Biomedical Research Institute (Rockville, MD). Three weeks after the final immunization mice were challenged with 150 cercaria via tail exposure and sacrificed seven weeks later. Images of mouse livers were taken during dissection using a Galaxy S8 cell phone camera (Samsung Group, Seoul, South Korea). Adult worms were perfused from the hepatic portal system and counted manually (Ricciardi et al., 2016). Liver sections were collected for histology as previously described (Hassan et al., 2019A). Burden reductions were calculated as previously described (Ricciardi et al., 2015; Ricciardi et al., 2016; Hassan et al., 2019B).

Serum Total SmCB-specific IgG, IgG1, and IgG2c. SmCB-specific serum IgG, IgG1, and IgG2c was assessed by ELISA as described elsewhere (Hassan et al., 2019A). IgG1 and IgG2c endpoint titers were calculated as the reciprocal of the highest dilution which gave a reading above the cut-off. The endpoint titer cut-off was statistically established as described elsewhere (Frey et al., 1998) using the sera of PBS immunized, unchallenged mice.

Serum Total IgE. Total IgE was assessed by ELISA using the BD OptEIA™ Set Mouse IgE Kit (BD, San Diego, CA) following manufacturer's guidelines.

Cell-Mediated Immune Responses. After immunization, mice were sacrificed, spleens collected, and splenocytes isolated as previously described (Yam et al., 2015) with the following exceptions: splenocytes were resuspended in RPMI-1640 supplemented with 10% fetal bovine serum, 1 mM penicillin/streptomycin, 10 mM HEPES, 1×MEM non-essential amino acids, 1 mM sodium pyruvate, 1 mM L-glutamine (Wisent Bioproducts), and 0.05 mM 2-mercaptoethanol (Sigma Aldrich) (fancy RPMI, fRPMI). These cells were then used in the following assays:

Proliferation Assay by BrdU. Cell proliferation was measured by using the Roche chemiluminescent kit, following manufacturer's guidelines. Splenocytes were seeded in black 96-well flat bottom plates at 200 000 cells per well. Each sample was seeded unstimulated, stimulated with SmCB (2.5 μg/mL), and stimulated with concavalin A as a positive control.

Cytokine Production by multiplex ELISA. Splenocytes were incubated at 300 000 cells in 200 μL with SmCB in fRPMI (2.5 μg/mL recombinant protein). After 72 hours at 37° C.+5% CO₂, plates were centrifuged and supernatant collected and stored at −80° C. until analysis. Cell supernatants were assessed for the presence of 16 cytokines and chemokines (IL1-a, IL1-b, IL-2, IL-3, IL-4, IL-5, IL-6, IL-10, IL-12p70, IL-17, IFNy, TNFa, CCL2 (MCP-1), CCL3 (MIP-1a), CSF2 (GM-CSF), and CCL5 (RANTES) using Q-plex Mouse Cytokine—Screen (16-plex) multiplex ELISA following the manufacturer's guidelines (Quansys Biosciences, Logan, UT, USA). Sera were run in singlet.

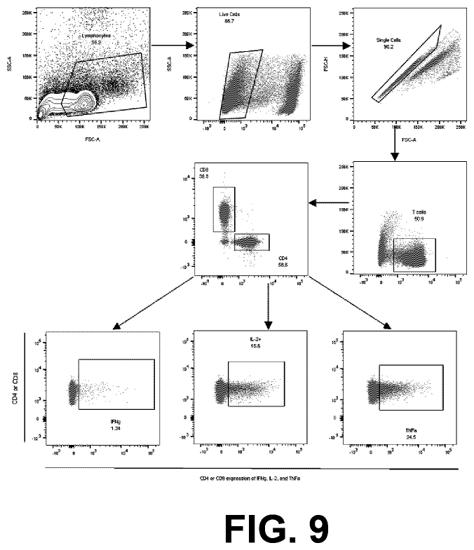

T Cell-Mediated Cytokine Secretion by Flow Cytometry. Splenocytes were seeded into 96-well U-bottom plates (BD Falcon) at 106 cells in 200 uL/well. Duplicate cultures were stimulated with or without SmCB in fRPMI (2.5 μg/mL) for 24 hours at 37° C.+5% CO₂. For the last 6 hours of incubation, protein transport inhibitor was prepared according to the manufacturer's guidelines (BD Science, San Jose, CA) and added to all samples. Cells stimulated with phorbol 12-myristate 13-acetate and ionomycin were processed as positive controls. Plates were then spun (350g, 8 minutes at 4° C.) and cells were processed for flow cytometry as described elsewhere (Hodgins et al., 2019). The following antibodies made up the extracellular cocktail: CD3-FITC (Clone 145-2C11, Affymetrix ebioscience), CD4-V500 (RM4-5, BD Bioscience) and CD8-PerCP-Cy5 (Clone:53-6.7, BD Science). The intracellular cocktail was made up of: IL-2-Pe-Cy5 (Clone: JES6;5H4, Biolegend, San Diego, CA), IFNγ-PE (Clone: XMG1.2, BD Science), and TNFα-efluor450 (Clone: MP6-XT22, Affymetrix ebioscience). After staining, cells were resuspended in PBS and analyzed on BD LSRFortessa X-20 (BD Science) using Flowjo software (version 10.0.8r1). The gating strategy is shown in FIG. 9.

Histology and Egg Granuloma Quantitation. Liver sections in 10% buffered formalin phosphate were stained using hematoxylin and eosin to assess granuloma size and egg morphology. Granuloma areas were measured using Zen Blue software (version 2.5.75.0; Zeiss) as previously reported (Hassan et al., 2019A; Cronan et al., 2018). Briefly, while working at 400x magnification, the pointer was used to trace the perimeter of 37-41 granulomas per experimental group with a clearly visible egg which the software converted into an area. Fifteen different fields of vision were assessed per experimental group and abnormal eggs were counted and reported as a percent of the total eggs counted.

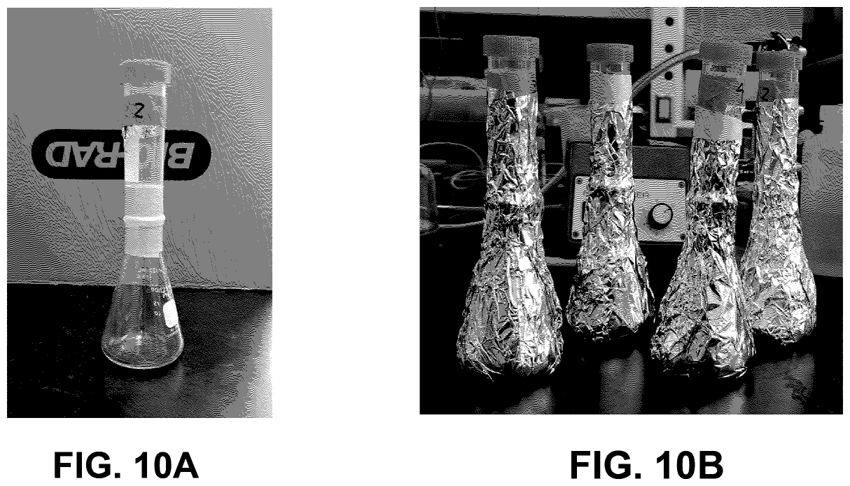

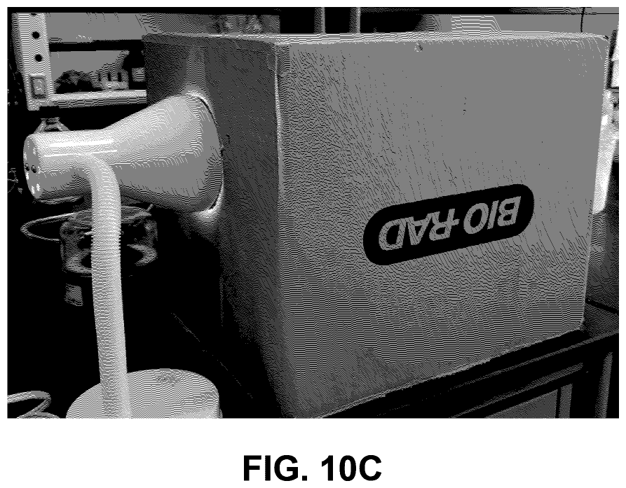

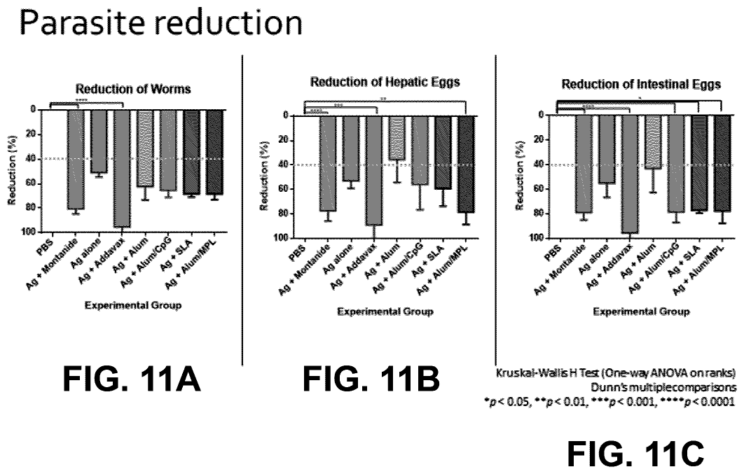

Miracidia Hatching Experiments. Miracidia hatching was optimized and adapted from a protocol as described elsewhere (Jurberg et al., 2008), see also FIG. 10. Briefly, seven weeks post challenge, one gram of feces was put into 15 mL conical tubes with distilled water. Fecal samples were homogenized then transferred to 125 mL Erlenmeyer flasks. The bottom sections of 50 mL conical tubes were removed using an exacto knife and attached tightly to the top of each Erlenmeyer flask using parafilm and tape. The Erlenmeyer flask and tube were covered in tin foil to protect from light, except for the top 3 mm of the tube under the twist-on cap. The flask and tube were then filled with distilled water to the top and the cap was put on. These light-protected chambers were put inside of a cardboard box, to further protect from light. A hole was cut into the cardboard box and a lamp was shone into it to direct light at the small sections on unprotected tube (3 mm under the cap). After 3 hours, the top 3 mm of water was removed from the tube using a pipette gun and ejected into a 12-well plate. Hatched miracidia were then counted using a dissecting microscope. An image of the set up can be seen in Supplemental FIG. 2.

Statistical Analysis. Statistical analysis was performed using GraphPad Prism 6 software (La Jolla, CA). Data were analyzed by Kruskal Wallis one-way ANOVA with Dunn's multiple comparisons tests. Flow cytometry data were analyzed by a two-way ANOVA and Dunnet's multiple comparisons tests. If present, outliers were calculated using GraphPad QuickCalcs and removed. P values<0.05 were considered significant.

Humoral Response to vaccination. No mice had detectable SmCB-specific IgG antibodies at baseline, and PBS control remained negative throughout the study. Mice receiving adjuvanted rSmCB developed SmCB-specific IgG after a single immunization. At week 3, groups adjuvanted with Montanide and AddaVax had significantly higher titers than with SLA, however this difference was no longer significant post first boost. Antigen specific IgG titers in vaccinated mice rose until week 6 before plateauing.

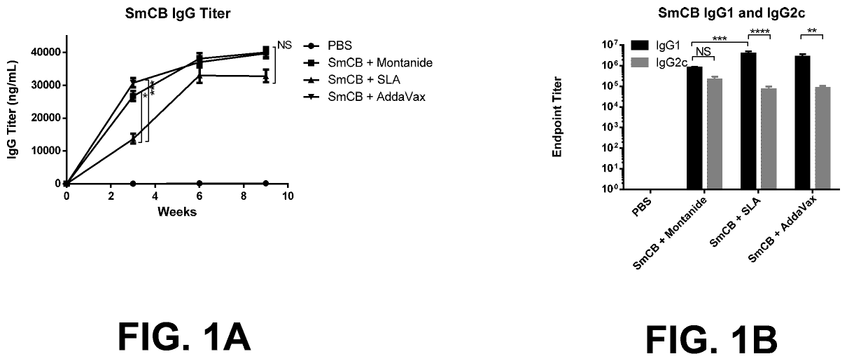

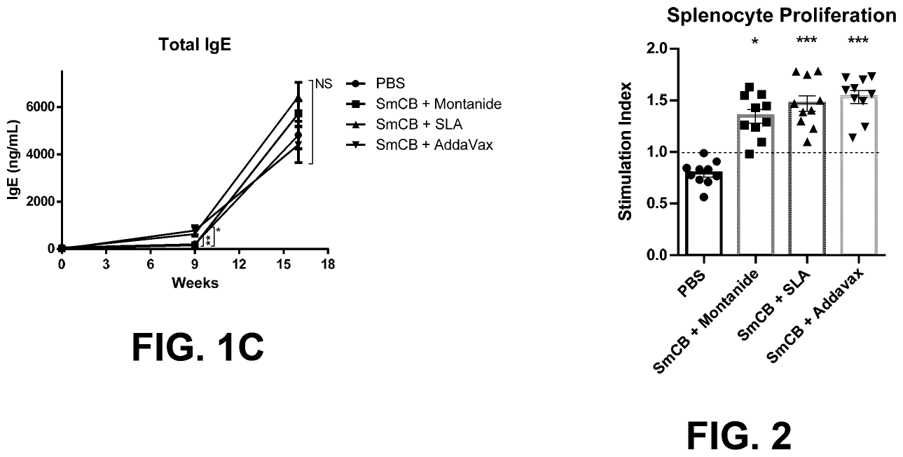

Endpoint titers were calculated for antigen specific IgG1 and IgG2c at the time of infection (FIG. 1B). Each experimental group elicited a robust mixed IgG1/IgG2c response, although mice vaccinated with antigen and SLA or AddaVax had much higher IgG1 (3.84e6±1.13e6 and 2.69e6±9.24e5 respectively) than IgG2c titers (6.60e4±3.21e4 and 7.88e4±2.38e4 respectively). Mice immunized with rSmCB/Montanide had a balanced IgG1/IgG2c response with titers of 8.00e5±1.09e5 and 2.03e5±9.75e4 respectively. At this time, and at the study endpoint (post-challenge) mouse serum was also analyzed for total IgE (FIG. 1C). At baseline, mice had little to no detectable IgE. In comparison to PBS controls, there was a greater increase in total IgE levels post immunization in groups receiving rSmCB adjuvanted with SLA (˜3 fold) or Addavax (˜3.5 fold) than in the group immunized with rSmCB adjuvated with Montanide which saw no increase. Upon parasite challenge, the total IgE titers increased in all groups including the PBS controls with no significant differences between groups.

Lymphoproliferation in response to vaccination. Enhanced SmCB-specific lymphoproliferation was seen in ex vivo stimulated splenocytes from immunized compared to control mice. However, no statistical differences in the magnitude of lympho-proliferation was observed between immunized groups (FIG. 2). Differences in functionality of antigen-specific lymphocytes were further assessed by measuring cytokine and chemokine concentrations in culture supernatants.

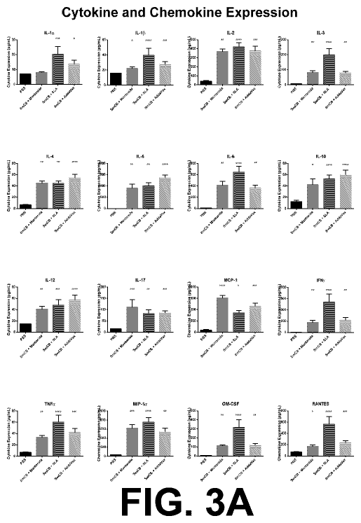

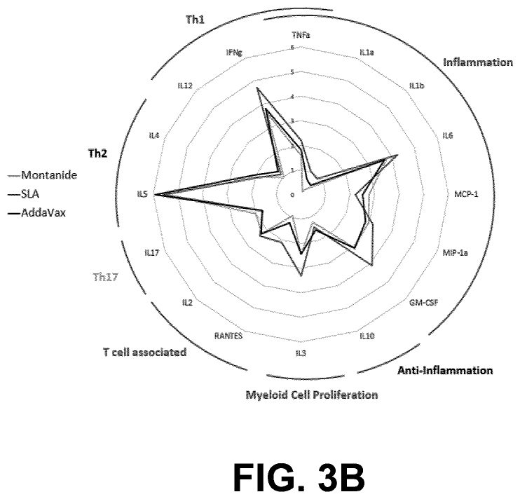

Splenocyte Cytokine and Chemokine Production in response to vaccination. For many of the cytokines and chemokines tested, adjuvanted formulations generated elevated levels above the PBS control (FIG. 3A). However, differences can be seen in the cytokine milieus between experimental groups. The fold change expression of each cytokine/chemokine from the PBS control is depicted in a radar plot (FIG. 3B) indicating each vaccine formulation favours a slightly different immune phenotype. Montanide has an increased Th17 immune profile, SLA an inflammatory, Th1, T-cell associated, and myeloid proliferating profile, and AddaVax a Th2 and anti-inflammatory profile.

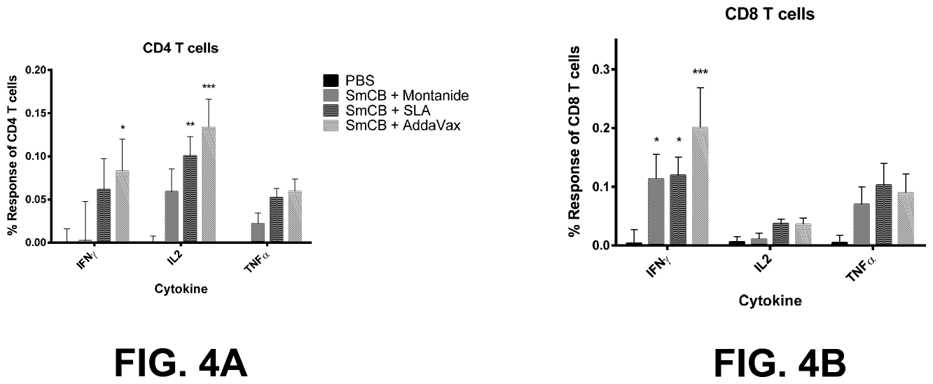

T cell Th1 response to vaccination. Flow cytometry was used to enumerate splenic CD4⁺ and CD8⁺ T cell expression of IFNγ, IL-2, and TNFα in response to SmCB. Overall, an increase cytokine expression were observed in CD4⁺ (FIG. 4A) and CD8⁺ (FIG. 4B) T cells in groups immunized with adjuvanted rSmCB over PBS controls. Mice immunized with rSmCB adjuvanted with SLA showed a significant increase in CD4⁺ IL-2 expression whereas mice immunized with rSmCB adjuvanted with AddaVax showed a significant increase in CD4⁺ IL-2 and IFNγ expression compared to PBS control mice. All groups receiving adjuvanted rSmCB showed a significant increase in CD8⁺ IFNγ expression.

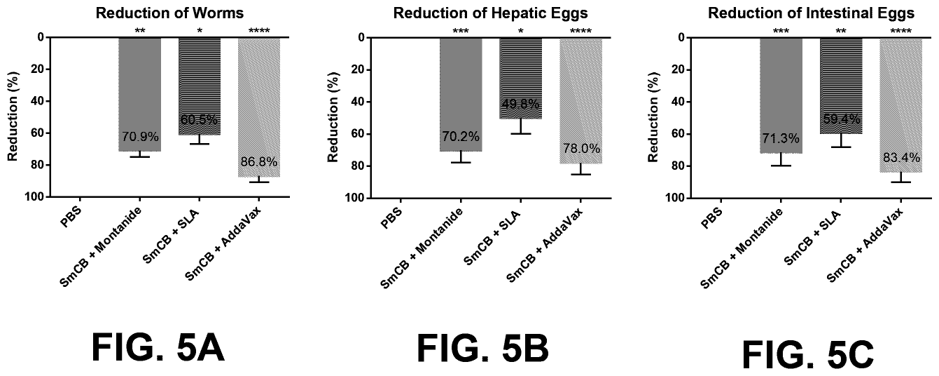

Protection from infection upon immunization with adjuvanted SmCB. To determine the protective potential of the vaccines, a three-dose immunization regiment was tested. The average amount of worms collected from PBS control mice was 31±7 worms over independent experiments. Parasite burden reductions were calculated in reference to the PBS control mice within the same experiment to keep consistency within batches of infections. Parasite burden reductions were then combined and compared. All vaccine formulations significantly reduced parasite burden over PBS control with percent reduction in worm burden of 70.9±3.9%, 60.5±6.3% and 86.8±4.0% in groups adjuvanted with Montanide, SLA and Addavax respectively (FIG. 5A). There were no statistical differences in worm burdens between the three formulations.

Pathology in schistosomiasis is caused by parasite eggs which become trapped in host tissues. Egg burdens in the liver (FIG. 5B) and intestines (FIG. 5C) were also calculated. Hepatic eggs in the PBS control group varied between 1250 and 14525 eggs/gram liver tissue. Similarly, intestinal eggs ranged between 1660 and 16973 eggs/gram intestine. rSmCB/Montanide reduced parasite burden by 70.3±7.4% and 71.3±8.4% in hepatic and intestinal eggs, respectively. The formulation of rSmCB/SLA reduced parasite burden by 49.8±9.9% and 59.4±8.8%, while rSmCB/AddaVax reduced parasite burden the most significantly, 78.0±7.2% and 83.4±6.6%, in hepatic and intestinal eggs respectively.

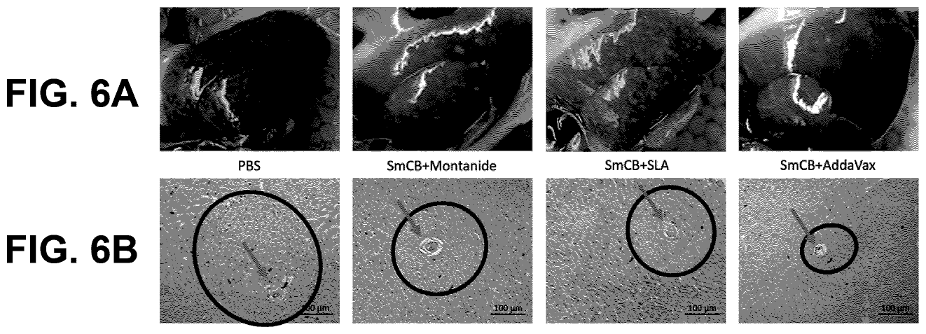

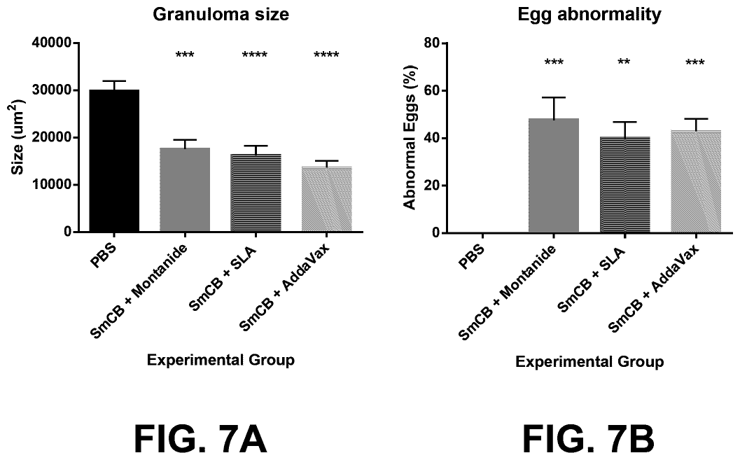

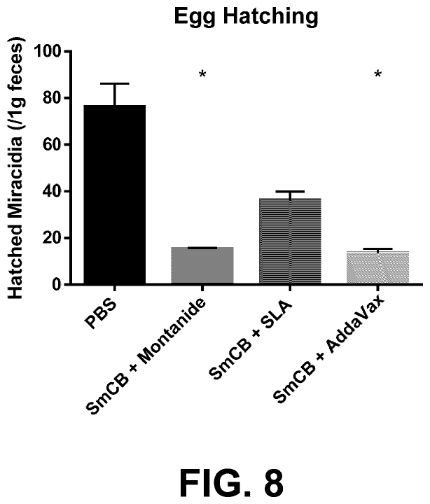

Liver pathology. During mouse dissection, images were taken of gross liver sections as pathology was clearly visible (FIG. 6A). Livers from PBS control mice had many granulomas (visualized as white circular formations) that covered the surface of the liver due to heavy egg deposition, while vaccinated mice in all groups had less granuloma formation compared to PBS controls. By visual examination, mice immunized with rSmCB adjuvanted with Montanide and AddaVax had the least granuloma formation. Microscopic examination of liver tissue stained with hematoxylin and eosin stain (FIG. 6B) revealed the presence of S. mansoni egg within granulomatous formations. Granulomas were large, and well formed in PBS control mice, and eggs in granulomas were intact with normal appearances. Upon vaccination with adjuvanted rSmCB, granuloma sizes dropped from approximately 30000 μm² to below 20000 μm² (FIG. 7A). Mean granuloma sizes in rSmCB formulated with Montanide and SLA were 17541±1991 μm² and 16185±2070 μm² respectively. Although granulomas were smallest in the group adjuvanted with AddaVax (13637±1398 μm²) there were no statistical differences between vaccinated groups. Eggs in vaccinated animals were also abnormal in appearance (ie: internal structure was lost or compromised, edges were crenellated and incomplete) (FIG. 7B). A percentage of 47.7±9.5% eggs were found to be abnormal in mice immunized with rSmCB/Montanide. When rSmCB was adjuvanted with SLA and AddaVax, 39.9±7.0% and 42.9±5.3% of eggs were found to be abnormal, again these differences were not significant.

Egg hatching. To assess whether our vaccine formulations could interrupt the transmission of schistosomiasis we tested whether eggs retrieved from feces were able to give rise to larvae. Feces from PBS control mice gave rise to 76.3±10.0 miracidia. Feces from experimental groups saw significant reductions in miracidia: 15.4±0.4, 36.2±3.7, and 13.6±1.7 miracidia hatched from Montanide, SLA, and AddaVax groups respectively, with no statistical significance between them.

It was previously shown the protective capabilities of SmCB, when adjuvanted with CpG dinucleotides (Ricciardi et al., 2015) and Montanide ISA 720 VG (Ricciardi et al., 2016). In the present example, the protective capabilities of two new adjuvants: sulfated lactosyl archaeol (SLA) archaeosomes and AddaVax, a squalene based oil-in-water emulsion similar to MF59® were evaluated. When used as an adjuvant, SLA has been shown to activate strong humoral and cell-mediated responses against multiple antigens by increasing local cytokine production, immune cell trafficking, and antigen uptake at the injection site, leading to increased protection in murine models of infectious disease and cancer. In the present example, a novel admixed formulation was used and provided a simple ready to mix adjuvant formulation with no loss of antigen during formulation process. AddaVax alternatively, is a squalene-oil based emulsion structurally similar to MF-59. which acts by stimulating local cytokine and chemokine production, attracting immune cells to the injection site and increasing antigen trafficking and presentation.

SmCB, is a gut cysteine peptidase necessary for parasite growth and maturity. Although immunogenic and capable of protecting from S. mansoni infection when used alone (Tallima et al., 2017), it was previously shown that adjuvants enhance its immunogenicity and protective efficacy (Ricciardi et al., 2015; Ricciardi et al., 2016; Ricciardi et al., 2018), the highest protection seen with Montanide (Ricciardi et al., 2016). The two novel adjuvanted formulations in the present example were able to surpass the WHO schistosomiasis vaccine threshold of 40% protection. SLA reduced adult worms, liver eggs, and intestinal eggs by 60.5%, 49.8%, and 59.4% respectively, while AddaVax reached 86.8%, 78.0%, and 83.4% in the same readouts (FIG. 5).

Eggs trapped in host tissues release soluble egg antigens triggering granuloma formation, leading to liver cirrhosis and other fatal morbidities. Both emulsion-based vaccines (Montanide and AddaVax) were able to visibly reduce granuloma size, and parasite pathology to the liver (FIG. 6). Granuloma formation is initiated by Th2 immune responses however when mice mount extreme Th1 polarization responses, liver pathology is severe. This was shown in mice immunized with schistosome egg antigens (SEA) and complete Freunds adjuvant, and again in mice that lack both IL-10 and IL-4 which reached 100% mortality upon infection with schistosomiasis. Although SmCB is not expressed by eggs trapped in host tissue, it is a secreted protein of the adult fluke which resides in venules in and around the liver and intestines. It is possible that SmCB specific lymphocyte reactivation is causing the expression of Th1 and inflammatory cytokines that are indirectly contributing to the deleterious liver pathology seen in SLA vaccinated animals. Despite a greater number of eggs found in SLA liver tissues than Montanide and AddaVax, granulomas around these eggs were equally reduced in size.

Eggs released into freshwater in feces, will hatch miracidia, the first larval stage of the parasite. It was found that one gram of feces led to a reduced number of hatched miracidia in animals vaccinated with SmCB and Montanide or AddaVax. As shown in previous work (Correnti et al., 2005; Morales et al., 2008), targeting a digestive enzyme may lead to a suppression of metabolic activities necessary for proper reproduction, leading to the lowered fertility and egg fitness demonstrated by the vaccines of the present example.

Immunogenicity studies suggest that the protection mediated by the vaccine formulations could be explained by a robust humoral and cellular mediated immunity (CMI) and it is likely that both these responses contribute to protection from schistosomiasis.

Several groups have shown a positive correlation between IgG antibody titer and protection from schistosomiasis suggesting a necessity for the humoral response. This response was seen to mediate antibody mediated cellular cytotoxicity (ADCC) and activate complement as an attack against schistosomula in vitro. By this mechanism or due to another, high IgG titers have been found in vaccinated animals with reduced adult worm burdens. Interestingly, a study in rhesus macaques, not only showed a reduction in worm burden, but the worms collected were morphologically stunted with degenerated reproductive systems. As the vaccine formulations characterized in this example produced robust IgG titers, they all showed promise for a protective vaccine. Learning from the failed hookworm vaccine, it was sought to ensure the vaccine formulations did not cause IgE hypersensitivity, as IgE is a trademark of helminth infections like S. mansoni. A slight increase in total IgE levels was observed after immunization using SLA and AddaVax which were not present in Montanide adjuvanted groups or the PBS control. However, post challenge, total IgE levels were similar in all groups including unvaccinated controls (FIG. 1C). Thus, detrimental effects associated with vaccine induced IgE responses in unlikely.

Ex vivo re-stimulation of splenocytes with rSmCBshowed significant lymphoproliferation in all vaccinated groups, so it was determined what cell mediated immunity was being elicited by the different vaccines. Although all vaccine groups increased cytokine expression, there were subtle differences in their cytokine milieus between different adjuvant formulations (FIG. 3B). When combined with SLA, SmCB was broadly stimulating increasing inflammatory cytokines, Th1 and T-cell associated cytokines, as well as the myeloid proliferation cytokine IL-3, whereas with Montanide and AddaVax, SmCB led to increased Th17, and Th2/Anti-inflammatory cytokines respectively.

From the creation of the S. mansoni radiation-attenuated cercaria vaccine, it has been the consensus that IFNγ and TNFα play pivotal roles in protection. It is a promising feature that when CD4⁺ and CD8⁺ T cells from vaccinated animals were stimulated ex vivo with SmCB we observed increases of IFNγ, with trends of increased TNFα. Although the percentage increases observed are small, the number of cells that they represent specific to our antigen is significant. Interestingly, the multiplex ELISA data shows significant production of IFNγ by all vaccinated groups, which is not be fully reflected in the T cell expression as seen by flow cytometry. Future studies could prove useful to identify which other cell types are contributing to IFNγ expression, especially for mice vaccinated with Montanide and SLA.

Previous work has shown 70% reduction in worm burden with Sm-p80 tegument vaccine administered by DNA prime and boosted with protein and oligodeoxydinucleotides (Ahmad et al., 2009), and 57% reduction in worm burden Sm-Tsp-2 tetraspanin vaccine adjuvanted with Freund's incomplete adjuvant (Pearson et al., 2012), among others.

However, SLA archaeosomes and AddaVax in the presence of SmCB and are reducing adult worm burden the most significantly of all the recombinant protein vaccines in pre-clinical trials. The data supports the hypothesis that Schistosoma mansoni cathepsin B is a strong candidate for an anti-schistosome vaccine and can be readily formulated with multiple different types of adjuvants including oil-in-water and water-in-oil emulsions, archaeosomes and TLR9 agonists. Future directions include conducting dose response experiments on tested adjuvants, as a single dose level of SLA was tested and AddaVax was formulated as per the manufacturer's guidelines. Additionally, it would be useful to conduct more in-depth immunological and mechanistic studies to further elucidate the correlates of protection being elicited by our vaccines.

### EXAMPLE II

Ethics statement. All animal procedures were performed in accordance with Institutional Animal Care and Use Guidelines approved by the Animal Care and Use Committee at McGill University.

Generation of Adenovirus (Ad) expressing SmCB (AdSmCB) vector. The AdSmCB was developed following a similar protocol as described in Haq et al. 2019. Briefly, the SmCB gene cassette combined a kozak sequence with the full length of SmCB (Genbank accession number M21309.1) followed by a proline-linked 6×histidine tag and the poly-A signal “AATAAAATATCTTTATTTTCATTACATCTGTGTGTTGGTTTTTTGTGTG” (SEQ ID NO: 1, GenScript, Piscataway, NJ, USA) (FIG. 1) and was synthesized and codon optimized to mouse and human expression by Integrated DNA Technologies (Coralville, IA, USA) and cloned into pShuttle-CMV-Cuo vector. This non-replicating and non-disseminating Ad (ΔE1, ΔE3; 1^{st} generation) encoding the S. mansoni Cathepsin B gene were made by homologous recombination in AdEasier-1 cells (strain), a gift from Bert Vogelstein (Addgene plasmid #16399). The resulting plasmid was linearized with PacI and transformed into HEK293A cells. Recombinant adenovirus was then amplified using SF-BMAd-R cells and purified by ultracentrifugation on CsCI gradients.

Western Blot assays. Western blot analysis to determine protein expression of SmCB by AdSmCB was performed after infection of HEK293A cells. Briefly, cells were infected and incubated for 24 hours followed by the lysis of cells using Lysis Buffer (0.1 M Tris, 10 μL EGTA, 50 μL Triton-100, 0.1 M NaCl, 1 mM EDTA, 25 μL 10% NaDeoxycholate, 1×protease inhibitor, in ddH₂O). Cell supernatants and lysates were resolved on a SDS-PAGE gel under reducing conditions, followed by transfer onto a nitrocellulose membrane then blocked with 5% milk (Smucker Foods of Canada Corp, Markham, ON, Canada), and 0.05% Tween 20 in phosphate buffered saline (PBS) (Fisher Scientific, Ottawa, ON, Canada) (PBS-T). The membrane was then incubated with mouse monoclonal anti-polyHistidine (Sigma Aldrich, Oakville, ON, Canada) antibody diluted 1:5 000 in PBS-TM overnight at 4° C. The membrane was then washed in PBS-T before incubation with horseradish peroxidase (HRP)-conjugated anti-mouse IgG (Sigma Aldrich) diluted 1:20 000 in PBS-T for one hour at room temperature. After incubation the membrane was washed again and developed using SuperSignal West Pico Plus Chemiluminescent Substrate (ThermoFisher Scientific, Waltham, MA, USA).

S. mansoni Cathepsin B Recombinant Protein Preparation (SmCB). S. mansoni Cathepsin B was prepared and purified as previously described in Ricciardi et al., 2015. Briefly, the PichiaPink™ system (Thermo Fisher Scientific) was used and recombinant yeast cells were cultured in a glycerol medium. After three days of growth, yeast cells were induced in a methanol medium to allow expression of recombinant protein. Recombinant protein was purified by Ni-NTA chromatography (Ni-NTA Superflow by QIAGEN, Venlo, Limburg, Netherlands). The elute was analyzed by Western Blot using antibodies directed at the His-tag.

Immunization Protocol. Six- to eight-week-old female C57BL/6 mice were bred from mice obtained from Charles River Laboratories (Senneville, QC). Four groups of mice (n=8) were immunized for humoral and cell mediated immunity assessment. Four groups of mice (n=8) were immunized and subsequently infected for parasite burden assessment, with an additional fifth group.

Group 1 (PBS): mice were injected with PBS (Wisent Bioproducts, St. Bruno, QC).

Group 2 (SmCB): mice were immunized with 20 μg of recombinant SmCB (SmCB) three times.

Group 3 (AdNeg: SmCB): mice were immunized with 10⁵ infectious units of an empty ΔE1ΔE3 adenovirus containing no gene cassette, followed by two boosts of 20 μg recombinant SmCB.

Group 4 (AdSmCB:SmCB): mice were immunized with 10⁵ infectious units of AdSmCB, followed by two boosts of 20 μg recombinant SmCB.

Group 5 (AdNeg): mice were immunized with 10⁵ infectious units of an empty ΔE1ΔE3 adenovirus containing no gene cassette, followed by two boosts of PBS.

Each mouse was immunized at weeks 0, 3, and 6 intramuscularly in the thigh with 50 μL of the vaccine preparation.

Schistosoma mansoni challenge. Biomphalaria glabrata snails infected with the Puerto Rican strain of S. mansoni were provided by NIAID Schistosomiasis Resource Center of the Biomedical Research Institute (Rockville, MD). At week 9, mice were challenged with 150 cercaria via tail exposure for one hour and sacrificed seven weeks later for parasite burden. Images of mouse livers were taken during dissection using a Galaxy S10 cell phone camera (Samsung Group, Seoul, South Korea). Adult worms were perfused from the hepatic portal system and counted manually. Liver sections were suspended in 10% buffered formalin phosphate (Fisher Scientific) and processed for histology as described before (Perrera et al., 2020; Hassan et al., 2019B). Remaining liver and intestines were weighed and digested overnight in 4% potassium hydroxide. The following day, eggs present in these tissues were counted by microscopy and adjusted per gram of tissue. Burden reductions were calculated as previously described (Perrera et al., 2020; Hassan et al., 2019B):

$$( 1 - \frac{\begin{matrix}( mean number of worm or eggs recovered \\ in immunized mice )\end{matrix}}{\begin{matrix}( mean number of worm or eggs recovered \\ in immunized mice )\end{matrix}} ) \times 100 \%$$

Serum Total SmCB-specific IgG, IgM, IgE, and IgG avidity. SmCB-specific serum IgG was assessed by ELISA as described elsewhere (Hassan et al., 2019B). Briefly, high binding 96-well plates (Greiner Bio-One, Frickenhausen Germany) were coated with recombinant Cathepsin B (0.5 μg/mL) in 100 mM bicarbonate/carbonate buffer (pH 9.6) overnight at 4° C. After blocking plates with 2% bovine serum albumin (BSA; Sigma Aldrich) in PBS-T (blocking buffer) serum samples were added to the plates in duplicate. For IgG, an additional set of samples were run in duplicate to determine avidity. Plates were incubated for one hour at 37° C. then washed with PBS (pH 7.4). For avidity testing, blocking buffer was added to the standard curve and one set of samples in duplicate, while the other set received 10 M urea and were covered and incubated for 15 minutes at room temperature before being washed four times and incubated another hour with blocking buffer. Then plates were washed another four times and anti-mouse IgG-HRP (Sigma Aldrich) was diluted 1:20 000 in blocking buffer and applied. For IgM and IgE, HRP-conjugated anti-mouse IgM (SouthernBiotech, Birmingham, AL, USA) or IgE (Thermofisher) was diluted 1:6000 in blocking buffer and applied. Again, plates were washed with PBS and 3,3′,5,5′-Tetramethyl benzidine (TMB) substrate (Millipore, Billerica, MA) was used for detection followed by the addition of H2SO4 (0.5M; Fisher Scientific). Optical density (OD) was measured at 450 nm with an EL800 microplate reader (BioTek Instruments Inc., Winooski, VT), and concentration of SmCB specific IgG was calculated by extrapolation from the IgG standard curve. IgG avidity index was calculated by dividing the IgG binding in the urea condition by the amount of IgG in the other condition. IgM and IgE were reported as OD values.

Serum SmCB-specific IgG1, and IgG2c. SmCB-specific serum IgG1, and IgG2c were assessed by ELISA as described elsewhere (Perrera et al., 2020; Hassan et al., 2019B). Briefly, Immunolon 2HB flat-bottom 96-well plates (Thermo Fisher) were coated with recombinant SmCB (0.5 μg/mL) in 100 mM bicarbonate/carbonate buffer (pH 9.6). Plates were washed with PBS-T and blocking buffer was applied for 90 minutes. A serial dilution of serum was applied to plates in duplicate and incubated for 2 hours at 37° C. Plates were washed again with PBS-T, and goat anti-mouse IgG1-HRP (SouthernBiotech) or goat anti-mouse IgG2c-HRP (SouthernBiotech) was applied to plates for one hour at 37° C. After a final wash, TMB was added followed by H₂SO₄. Again, OD was measured as above. IgG1 and IgG2c endpoint titers were calculated as the reciprocal of the highest dilution which gave a reading above the cut-off. The endpoint titer cut-off was statistically established as described elsewhere (Frey et al., 1998) using the sera of PBS immunized, unchallenged mice.

Cell-Mediated Immune Responses. Three weeks after the last immunization, mice were sacrificed, spleens were collected, and splenocytes were isolated as previously described (Yam et al., 2015) with the following exceptions: splenocytes were resuspended in RPMI-1640 supplemented with 10% fetal bovine serum, 1 mM penicillin/streptomycin, 10 mM HEPES, 1×MEM non-essential amino acids, 1 mM sodium pyruvate, 1 mM L-glutamine (Wisent Bioproducts), and 0.05 mM 2-mercaptoethanol (Sigma Aldrich) (fancy RPMI, fRPMI). These cells were then used in the following assays:

Cytokine Production by multiplex ELISA. Splenocytes were incubated at 1 000 000 cells in 200 μL with SmCB in fRPMI (2.5 μg/mL recombinant protein). After 72 hours at 37° C.+5% CO₂, plates were centrifuged and supernatant collected and stored at −80° C. until analysis. Cell supernatants were assessed for the presence of 16 cytokines and chemokines (IL1-a, IL1-b, IL-2, IL-3, IL-4, IL-5, IL-6, IL-10, IL-12p70, IL-17, IFNγ, TNFα, CCL2 (MCP-1), CCL3 (MIP-1a), CSF2 (GM-CSF), and CCLS (RANTES) using Q-plex Mouse Cytokine—Screen (16-plex) multiplex ELISA following the manufacturer's guidelines (Quansys Biosciences, Logan, UT, USA). Samples were run in singlet.

T Cell-Mediated Cytokine Secretion by Flow Cytometry. Splenocytes were seeded into 96-well U-bottom plates (BD Falcon) at 106 cells in 200 μL/well. Duplicate cultures were stimulated with or without SmCB in fRPMI (2.5 μg/mL) for 24 hours at 37° C.+5% CO₂. For the last 6 hours of incubation, protein transport inhibitor was prepared according to the manufacturer's guidelines (BD Science, San Jose, CA) and added to all samples. Cells stimulated with phorbol 12-myristate 13-acetate and ionomycin were processed as positive controls. Plates were then processed for flow cytometry as described elsewhere (Hodgins et al., 2019). Briefly, splenocytes were washed twice with 200 μL of cold PBS, and fixable viability dye eFluor 780 (Affymetrix ebioscience, Waltham, MA) was applied at 50 μL/well diluted at 1:300 and incubated for 20 minutes at 4° C. protected from light. Cells were washed as above with PBS 1% BSA (PBS-BSA), and Fc block (BD Science) diluted 1:50 was added for 15 minutes. All surface stains were diluted 1:50 in PBS-BSA and 50 μL/well of extracellular cocktail was applied for 30 minutes at 4° C. protected from light. The following antibodies made up the extracellular cocktail: CD3-FITC (Clone 145-2C11, Affymetrix ebioscience), CD4-V500 (RM4-5, BD Bioscience) and CD8-PerCP-Cy5 (Clone:53-6.7, BD Science). Cells were then washed as above with 1X fixation buffer (BD Science) and left overnight at 4° C. in the dark. Plates were washed as before with 1×permeabilization buffer (BD Science) and stained with an intracellular cocktail of antibodies diluted 1:50 in PBS-BSA applied as 50 μL/well for 30 minutes at 4° C. protected from light. The intracellular cocktail was made up of: IL-2-Pe-Cy5 (Clone: JES6;5H4, Biolegend, San Diego, CA), IFNγ-PE (Clone: XMG1.2, BD Science), and TNFα-efluor450 (Clone: MP6-XT22, Affymetrix ebioscience). After staining, cells were resuspended in PBS and analyzed on BD LSRFortessa X-20 (BD Science) using Flowjo software (version 10.0.8r1).

Histology and Egg Granuloma Quantitation. Liver sections in 10% buffered formalin phosphate were stained using hematoxylin and eosin to assess granuloma size and egg morphology. Granuloma areas were measured using Zen Blue software (version 2.5.75.0; Zeiss) as previously reported (3, 10, 14-17). Briefly, while working at 400× magnification, the pointer was used to trace the perimeter of 24-32 granulomas per experimental group in an exudative-productive stage with a clearly visible egg which the software converted into an area. Hepatic eggs were classified as abnormal if their internal structure was lost or the perimeter of the egg was crenelated. Eighteen to 32 different fields of vision were assessed per experimental group over two independent experiments. Abnormal eggs were counted and reported as a percent of the total eggs counted per field of vision.

CMI Dose Response. Pilot study comparing four doses of AdSmCB to determine cellular mediated immunity. Six- to eight-week-old female C57BL/6 mice were immunized for parasite burden reduction. Group 1: control: mice were injected with phosphate-buffered saline (PBS) (Wisent Bioproducts, St. Bruno, QC). Group 2: AdVNeg: mice were immunized with 10\hat{ }9 infectious units of empty adenovirus vector and boosted twice with 20 μg SmCB. Groups 3-6: mice were immunized with 10\hat{ }5, 10\hat{ }7, 10\hat{ }9, or 5*10\hat{ }9 infectious units of recombinant adenovirus vector and boosted twice with 20 μg SmCB. Each mouse was immunized at weeks 0, 3, and 6 intramuscularly in the thigh with 50 μL of vaccine. Mice were sacrificed at week 9 and splenocytes were harvested and restimulated with SmCB before being analyzed by flow cytometry as described in our paper. Data are shown as mean and SEM. N=5.

Statistical Analysis. Statistical analysis was performed using GraphPad Prism 6 software (La Jolla, CA). Data were analyzed by Kruskal Wallis one-way ANOVA with Dunn's multiple comparisons tests. Flow cytometry data were analyzed by a two-way ANOVA and Dunnett's multiple comparisons tests. If present, outliers were calculated using GraphPad QuickCalcs and removed. P values<0.05 were considered significant.

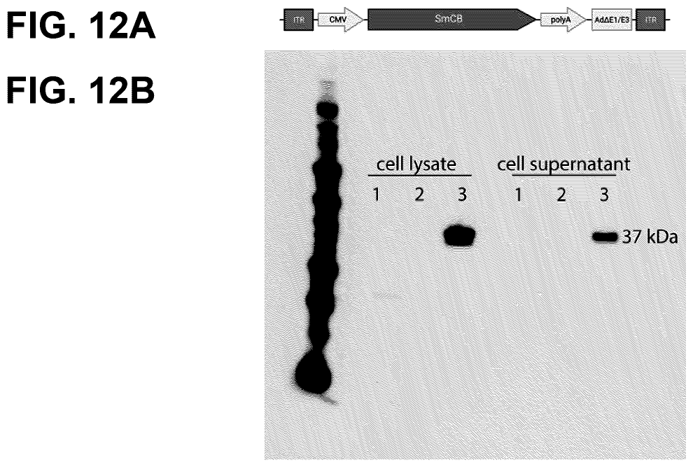

A Western blot directed against the histidine tag of SmCB was run to determine the expression and secretion of SmCB by the recombinant adenovirus (see genetic construct in FIG. 12A). The presence of SmCB was analyzed in the cell lysates and culture medium (FIG. 12B).

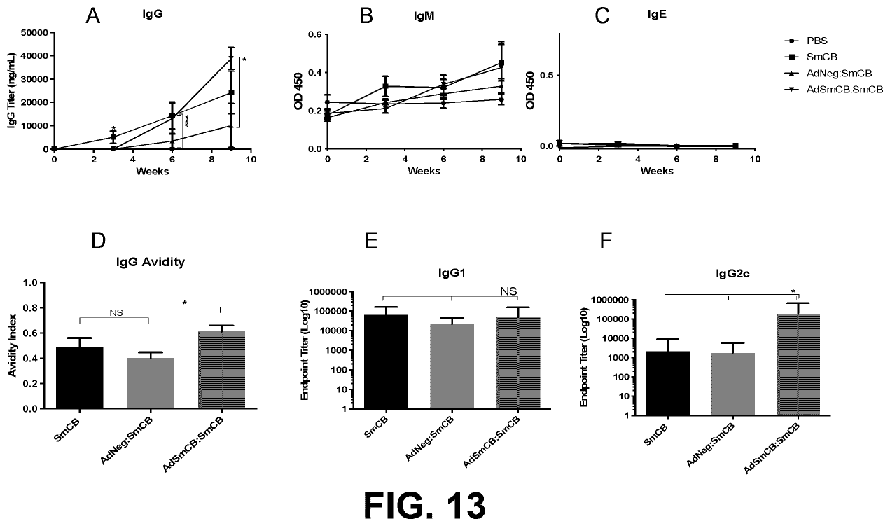

Humoral responses were determined throughout the immunization schedule. No mice had detectable SmCB specific IgG, IgM, or IgE at baseline, and the PBS control remained negative throughout the study. Mice receiving SmCB developed antibody titers after a single immunization, whereas mice receiving both empty or recombinant Ad only developed antibodies after the first boost. By week 6, IgG titers between SmCB and AdSmCB:SmCB groups were no longer significantly different, however at the end of the immunization period AdSmCB:SmCB produced significantly higher titers than the AdNeg:SmCB group (FIG. 13A). Over the course of the immunization period, animals in the SmCB, AdNeg:SmCB, and AdSmCB:SmCB groups, saw a trend of increasing antigen specific IgM however this trend was not significant (FIG. 13B). Finally, no animals developed antigen specific IgE in response to vaccination (FIG. 13C). The avidity of antigen specific IgG (FIG. 13D) and IgG subtype was determined at the time of infection. All vaccinated animals which developed IgG antibodies developed highly avid antibodies. Although there was no significant difference between those antibodies developed in SmCB vaccinated animals and AdSmCB:SmCB, the priming recombinant Ad increased avidity when compared to its empty Ad counterpart. There were no statistical differences between the amount of IgG1 produced by any of the experimental groups (FIG. 13E), however AdSmCB:SmCB significantly increased the production of SmCB specific IgG2c (3.66e5±1.39e5) when compared to both the SmCB (9641±7775) and AdNeg:SmCB groups (3047±904.5) (FIG. 13F).

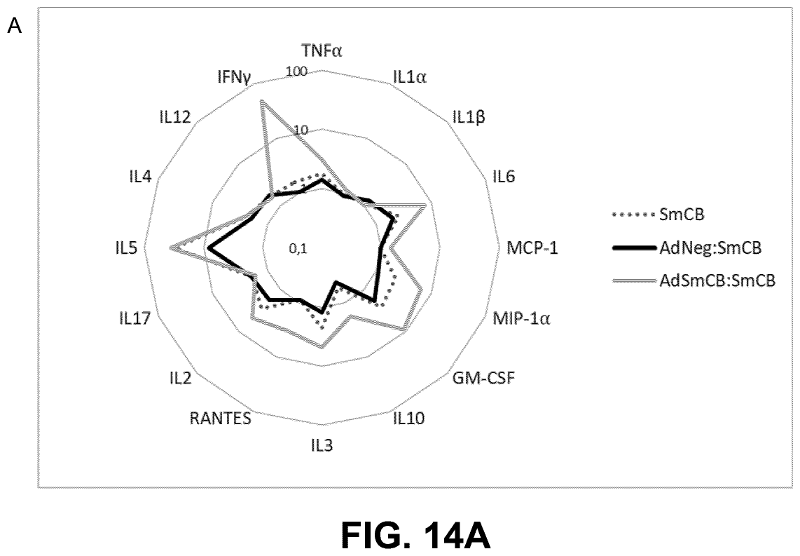

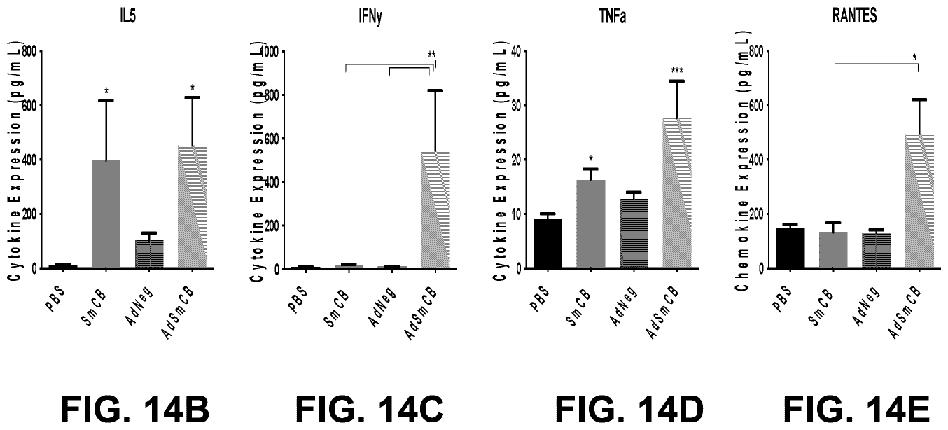

To determine the immune landscape of lymphocyte responses created by vaccination, a multiplex ELISA was ran on the supernatants of stimulated splenocytes. For many of the cytokines and chemokines tested the AdSmCB:SmCB group generated elevated levels of molecular signals shown in the radar plot (FIG. 14A). Notably, AdSmCB:SmCB maintains significant expression of IL5 also seen in the SmCB group (FIG. 14B), while enhancing expression of IFNγ (FIG. 14C), TNFα (FIG. 14D), and RANTES (FIG. 14E).

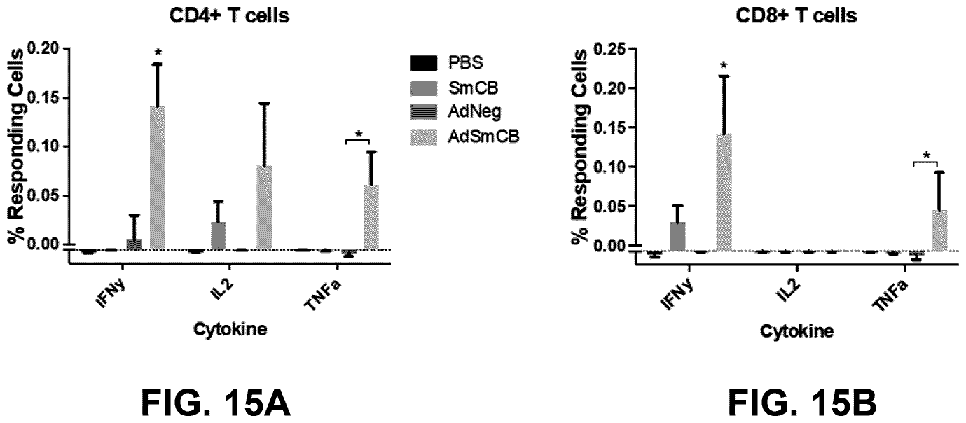

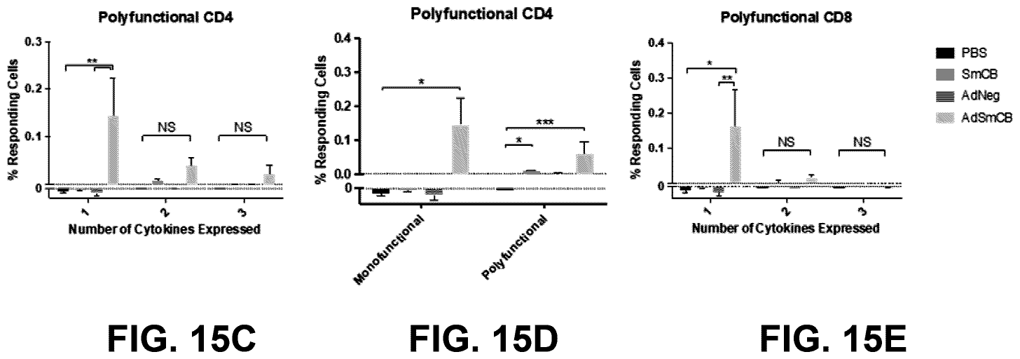

As a key contributor of protection in Schistosoma radiation attenuated vaccine models, the increased expression of IFNγ was further characterized. To determine if T cells were responsible for IFNγ expression in our recombinant Ad group, flow cytometry analysis was conduected. Indeed, when splenic T cells were stimulated ex vivo with antigen, an increased number of responding CD4+ (FIG. 15A) and CD8+ (FIG. 15B) T cells expressing IFNγ and TNFα was observed. Using a Boolean analysis, it was determined whether these cells were monofunctional or polyfunctional. It was found that recombinant Ad CD4+ and CD8+ T cells were largely monofunctional (FIGS. 15C and 15E). However, when cells expressing 2 and 3 cytokines were combined, an increase in CD4+ polyfunctionality in SmCB vaccinated animals was observed, and more so in animals immunized first with AdSmCB (FIG. 15D).

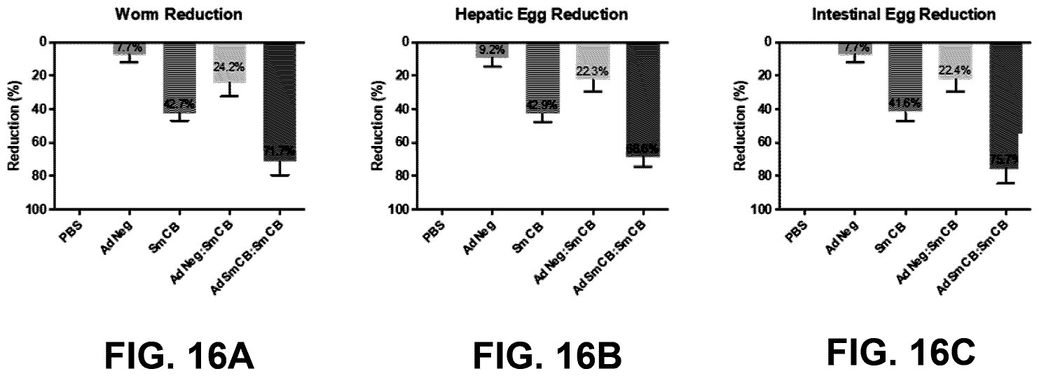

To determine the protective efficacy of the adenoviral vaccine, animals were infected and then sacrificed to determine adult worm, hepatic, and intestinal egg burden. These burdens were then compared to the PBS control to assess parasite burden reduction. A fifth group of mice, vaccinated with an empty Adenovirus vector with no boosting immunizations of protein, was included to ensure protective capacity could not be attributed non-specifically by the vector itself. The average amount of adult worms collected from control mice was 37±7 worms over two independent experiments, and reduction was calculated against the PBS control group of the same experiment to reduce batch discrepancy between infections. The AdNeg control group was unable to significantly reduce adult worm burden from the PBS control. However, when this empty vector was boosted twice with recombinant protein, protection increased to 24.2% (FIG. 16A). Worm burden was further reduced in animals vaccinated with three doses of recombinant protein, and those initially immunized with the recombinant Ad by 42.7% and 71.7% respectively (FIG. 16A). Egg deposition by adult worms is the main causative agent of pathology in schistosomiasis. Egg burdens in both livers (FIG. 16B) and intestines (FIG. 16C) were also calculated. Hepatic eggs ranged between 2588 and 30742 eggs per gram and intestinal eggs ranged between 2500 and 41135 eggs per gram of tissue in the PBS control. Similar to worm reduction, AdNeg was unable to confer any significant protection from egg deposition. Animals immunized with recombinant protein alone reduced liver and intestinal eggs by 42.9% and 41.6% respectively whereas animals immunized with recombinant Ad were protected from liver and intestinal eggs by 68.6% and 75.7% respectively.

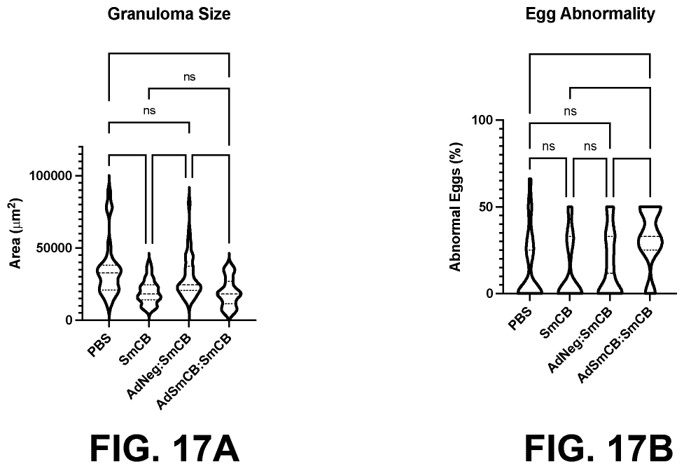

Microscopic examination of liver tissue stained by hematoxylin and eosin staining was used to assess granuloma formation after infection. Granulomas in control mice were large and well formed with an average of 34990.94±3818 μm² harboring intact eggs with normal appearances. When compared to both the PBS control and the AdNeg:SmCB groups, only recombinant protein (SmCB) and recombinant adenovirus (AdSmCB:SmCB) groups were able to reduce granuloma sizes to 19450.79±1477 μm² and 18898.04±2094 μm² respectively (FIG. 17A). Although the amount of abnormal eggs increased in all mice which received recombinant protein, only the AdSmCB:SmCB group showed a significantly increased proportion (FIG. 17B) reaching 32% compared to the 14% of the PBS control.

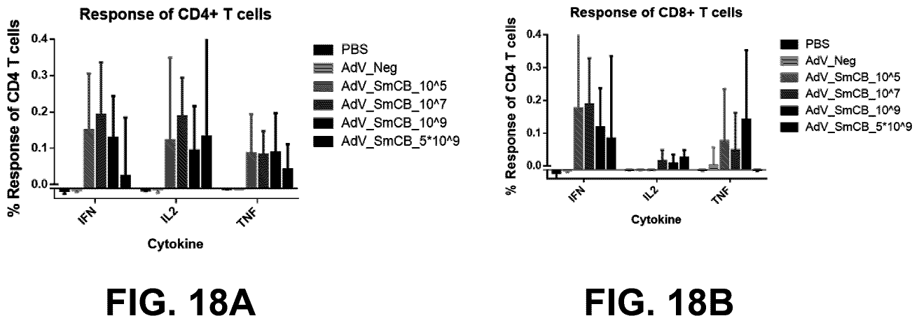

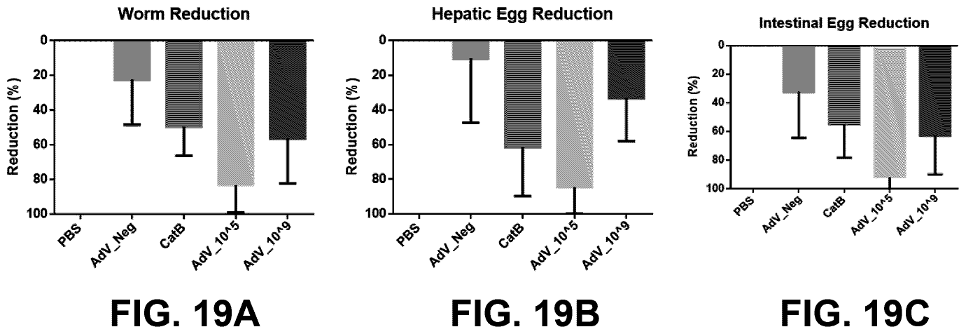

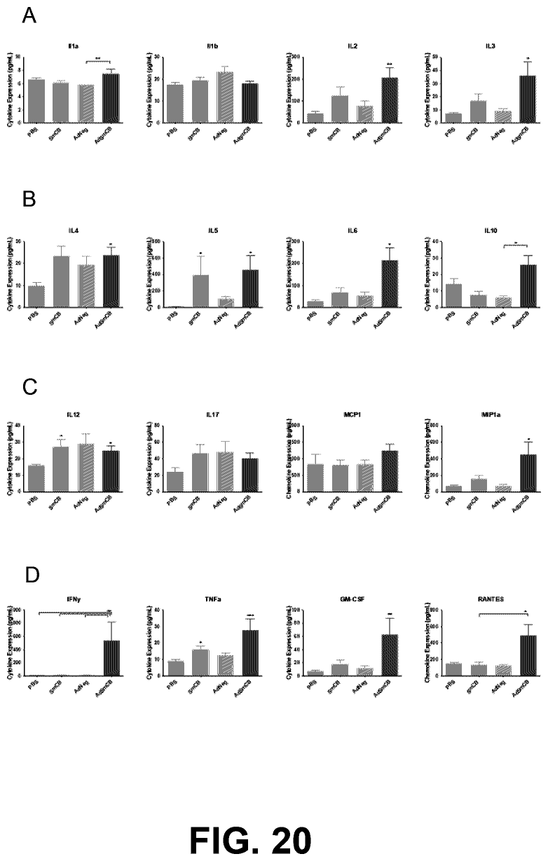

A pilot study was conducted to compare different doses of the Ad vaccine. The results are shown in FIGS. 18 and 19.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Correnti et al., 2005: Long-term suppression of cathepsin B levels by RNA interference retards schistosome growth. Mol Biochem Parasitol. Elsevier;143(2): 209-215.
Hassan et al., 2019A: Vaccination against the digestive enzyme Cathepsin B using a YS1646 Salmonella enterica Typhimurium vector provides almost complete protection against Schistosoma mansoni challenge in a mouse model. Yang R, editor. PLoS Negl Trop Dis
Ricciardi et al., 2015: Evaluation of the immune response and protective efficacy of Schistosoma mansoni Cathepsin B in mice using CpG dinucleotides as adjuvant. Vaccine. Elsevier Ltd; 2015; 33(2): 346-353.
Ricciardi et al., 2016: A vaccine consisting of Schistosoma mansoni cathepsin B formulated in Montanide ISA 720 VG induces high level protection against murine schistosomiasis. BMC Infect Dis. BioMed Central Ltd.;16(1).
Hassan et al., 2019B: Vaccination against the digestive enzyme Cathepsin B using a YS1646 Salmonella enterica Typhimurium vector provides almost complete protection against Schistosoma mansoni challenge in a mouse model. PLoS Negl Trop Dis
Frey et al., 1998: A statistically defined endpoint titer determination method for immunoassays. J Immunol Methods. 221(1-2): 35-41.
Yam et al., 2015: AS03-adjuvanted, very-low-dose influenza vaccines induce distinctive immune responses compared to unadjuvanted high-dose vaccines in BALB/c mice. Front Immunol. Frontiers Media S.A.; 6(APR).
Hodgins et al., 2019: A plant-derived VLP influenza vaccine elicits a balanced immune response even in very old mice with co-morbidities. PLoS One. Public Library of Science; 14(1).
Cronan et al., 2018: An explant technique for high-resolution imaging and manipulation of mycobacterial granulomas. Nat Methods. Nature Publishing Group; 15(12):1098-1107.
Jurberg et al., 2008: A new miracidia hatching device for diagnosing schistosomiasis. Mem Inst Oswaldo Cruz. Fundacao Oswaldo Cruz; 103(1):112-114.
Tallima et al., 2017: Protective immune responses against Schistosoma mansoni infection by immunization with functionally active gut-derived cysteine peptidases alone and in combination with glyceraldehyde 3-phosphate dehydrogenase. Fujiwara R T, editor. PLoS Negl Trop Dis
Ricciardi et al., 2018: Immune mechanisms involved in schistosoma mansoni-Cathepsin B vaccine induced protection in mice. Front Immunol. Frontiers Media S.A.; 9(July).
Morales et al., 2008: RNA interference of Schistosoma mansoni cathepsin D, the apical enzyme of the hemoglobin proteolysis cascade. Mol Biochem Parasitol [Internet]. Mol Biochem Parasitol; [cited 2020 Jun. 23]; 157(2):160-168.
Ahmad et al., 2009: Prime-boost and recombinant protein vaccination strategies using Sm-p80 protects against Schistosoma mansoni infection in the mouse model to levels previously attainable only by the irradiated cercarial vaccine. Parasitol Res.; 105(6):1767-1777.
Pearson et al., 2012: Enhanced Protective Efficacy of a Chimeric Form of the Schistosomiasis Vaccine Antigen Sm-TSP-2. Hirayama K, editor. PLoS Negl Trop Dis; 6(3):e1564.
Noya O, De Noya B A, Ballen D E, Bermúdez H, Bout D, Hoebeke J. Immunogenicity of synthetic peptides from the Sm31 antigen (cathepsin B) of the Schistosoma mansoni adult worms. Parasite Immunol. 2001;23(11):567-573.

Haq K, Jia Y, Elahi S M, MacLean S, Akache B, Gurnani K, et al. Evaluation of recombinant adenovirus vectors and adjuvanted protein as a heterologous prime-boost strategy using HER2 as a model antigen. Vaccine. 2019 Nov. 8;37(47):7029-40.

Perera D J, Hassan A S, Jia Y, Ricciardi A, McCluskie M J, Weeratna R D, et al. Adjuvanted Schistosoma mansoni-Cathepsin B With Sulfated Lactosyl Archaeol Archaeosomes or AddaVax™ Provides Protection in a Pre-Clinical Schistosomiasis Model. Front Immunol. 2020 Nov. 16;0:2990.

1. An immunogenic composition comprising an emulsion of an epitope, wherein:
the emulsion comprises an oil phase and a water phase;
the emulsion is an oil-in-water emulsion and/or a nanoemulsion; and
the epitope is present on a peptide or a polypeptide derived from Schistosoma sp. and can optionally be glycosylated.

2. The immunogenic composition of claim 1, wherein the oil phase comprises a squalene or a squalene derivative.

3. The immunogenic composition of claim 1 further comprising an emulsifier.

4. (canceled)

5. (canceled)

6. The immunogenic composition of claim 3, wherein the emulsifier is sorbitan trioleate.

7. The immunogenic composition of claim 1 further comprising a surfactant.

8. (canceled)

9. The immunogenic composition of claim 7, wherein the surfactant is a polysorbate.

10. (canceled)

11. The immunogenic composition of claim 1 further comprising a buffer.

12. (canceled)

13. The immunogenic composition of claim 11, wherein the buffer is a sodium citrate buffer.

14. The immunogenic composition of claim 1, wherein the peptide or the polypeptide is derived from Schistosoma mansoni.

15. The immunogenic composition of claim 1, wherein the peptide or the polypeptide is cathepsin B, an immunogenic cathepsin B variant or an immunogenic cathepsin B fragment.

16. The immunogenic composition of claim 15, wherein the peptide or the polypeptide is glycosylated.

17. An immunogenic composition comprising a nucleic acid molecule encoding cathepsin B, an immunogenic cathepsin B variant or an immunogenic cathepsin B fragment.

18. The immunogenic composition of claim 17, wherein the nucleic acid molecule is an adenovirus-derived vector.

19. (canceled)

20. The immunogenic composition of claim 17, further comprising a polypeptide, wherein the polypeptide comprises cathepsin B, an immunogenic cathepsin B variant or an immunogenic cathepsin B fragment.

21. (canceled)

22. A vaccine comprising the immunogenic composition of claim 1.

23. The vaccine of claim 22 being formulated for intramuscular or intranasal administration.

24. (canceled)

25. (canceled)

26. A method of preventing a Schistosoma sp. infection in a subject, the method comprising administering at least one dose of the immunogenic composition of claim 17 to the subject so as to prevent the Schistosoma sp. infection.

27. A method of treating a Schistosoma sp. infection or reducing a symptom associated with the Schistosoma sp. infection in a subject in need thereof, the method comprising administering at least one dose of the immunogenic composition of claim 17 to the subject so as to treat the Schistosoma sp. Infection, whereby the number of adult Schistosoma sp. worms is reduced in the subject, the number of Schistosoma sp. eggs is reduced in the subject, and/or the number of hatched Schistosoma sp. parasites is reduced in the stool of the subject, thereby reducing liver cirrhosis, liver granuloma formation and/or IgGE sensitivity in the subject.

28. (canceled)

29. (canceled)

30. (canceled)

31. (canceled)

32. The method of claim 26 for increasing a humoral response ora cell-mediated immune response against Schistosoma sp. in the subject.

33. (canceled)

34. (canceled)

35. (canceled)

36. The method of claim 26, wherein the Schistosoma sp. is Schistosoma japonicum, Schistosoma bovis, Schistosoma heamatobium, Schistosoma intercalatum, Schistosoma quineensis, Schistosoma curassoni, Schistosoma mattheei, Schistosoma mekonqi or Schistosoma mansoni.

37. A vaccine comprising the immunogenic composition of claim 17.

38. A method of preventing a Schistosoma sp. infection in a subject, the method comprising administering at least one dose of the immunogenic composition of claim 1 to the subject so as to prevent the Schistosoma sp. infection.

39. A method of treating a Schistosoma sp. infection or reducing a symptom associated with the Schistosoma sp. infection in a subject in need thereof, the method comprising administering at least one dose of the immunogenic composition of claim 1 to the subject so as to treat the Schistosoma sp. infection, whereby the number of adult Schistosoma sp. worms is reduced in the subject, the number of Schistosoma sp. eggs is reduced in the subject, and/or the number of hatched Schistosoma sp. parasites is reduced in the stool of the subject, thereby reducing liver cirrhosis, liver granuloma formation and/or IgGE sensitivity in the subject.
